(19) United States
(12) Patent Application Publication
(10) Pub. No.: US 2005/0261288 A1
(43) Pub. Date: Nov. 24, 2005

(54) Tetracyclic lactam derivatives and uses thereof

(76) Inventors: Prakash Jagtap, Beverly, MA (US); William Williams, Ipswich, MA (US); Csaba Szabo, Gloucester, MA (US)

Correspondence Address: WILMER CUTLER PICKERING HALE AND DORR LLP, 399 PARK AVENUE, NEW YORK, NY, 10022, US

(21) Appl. No.: 11/067,324

(22) Filed: Feb. 25, 2005

Related U.S. Application Data
(60) Provisional application No. 60/547,954, filed on Feb. 26, 2004.

(51) Int. Cl. A61K 31/5377; A61K 31/4745; C07D 471/02

(52) U.S. Cl. 514/233.2; 514/284; 544/125; 546/61; 546/70

(57) ABSTRACT

The present invention relates to Tetracyclic Lactam Derivatives, compositions comprising an effective amount of a Tetracyclic Lactam Derivative and methods for treating or preventing an inflammatory disease, a reperfusion injury, an ischemic condition, renal failure, diabetes, a diabetic complication, a vascular disease, reoxygenation injury resulting from organ transplantation, Parkinson's disease, or cancer, comprising administering to an animal in need thereof an effective amount of a Tetracyclic Lactam Derivative.

This application claims the benefit of U.S. Provisional Patent Application No. 60/547,954, filed Feb. 26, 2004, which is incorporated by reference herein in its entirety.

### 1. FIELD OF THE INVENTION

The present invention relates to Tetracyclic Lactam Derivatives, compositions comprising an effective amount of a Tetracyclic Lactam Derivative and methods for treating or preventing an inflammatory disease, a reperfusion injury, an ischemic condition, renal failure, diabetes, a diabetic complication, a vascular disease, reoxygenation injury resulting from organ transplantation, Parkinson's disease, or cancer, comprising administering to an animal in need thereof an effective amount of a Tetracyclic Lactam Derivative.

### 2. BACKGROUND OF THE INVENTION

Inflammatory diseases, such as arthritis, colitis, and autoimmune diabetes, typically manifest themselves as disorders distinct from those associated with reperfusion injuries, e.g., stroke and heart attack, and can clinically manifest themselves as different entities. However, there can be common underlying mechanisms between these two types of disorders. In particular, inflammatory disease and reperfusion injury can induce proinflammatory cytokine and chemokine synthesis which can, in turn, result in production of cytotoxic free radicals such as nitric oxide and superoxide. NO and superoxide can react to form peroxynitrite (ONOO⁻) (Szabó al., Shock 6:79-88, 1996).

The ONOO⁻-induced cell necrosis observed in inflammatory disease and in reperfusion injury involves the activation of the nuclear enzyme poly (ADP-ribose) polymerase (PARP). Activation of PARP is thought to be an important step in the cell-mediated death observed in inflammation and reperfusion injury (Szabó et al., Trends Pharmacol. Sci. 19:287-98, 1998).

A number of PARP inhibitors have been described in the art. See, e.g., Banasik et al., J. Biol. Chem., 267:1569-75, 1992, and Banasik et al., Mol. Cell. Biochem., 138:185-97, 1994; WO 00/39104; WO 00/39070; WO 99/59975; WO 99/59973; WO 99/11649; WO 99/11645; WO 99/11644; WO 99/11628; WO 99/11623; WO 99/11311; WO 00/42040; Zhang et al., Biochem. Biophys. Res. Commun., 278:590-98, 2000; White et al., J. Med. Chem., 43:4084-4097, 2000; Griffin et al., J. Med. Chem., 41:5247-5256, 1998; Shinkwin et al., Bioorg. Med. Chem., 7:297-308, 1999; Soriano et al., Nature Medicine, 7:108-113, 2001; and Southan and Szabo, Curr. Med. Chem., 10:321, 2003. Adverse effects associated with administration of PARP inhibitors have been discussed in Milan et al., Science, 223:589-591, 1984.

Synthesis and use of tetracyclic heterocyclic compounds have been previously discussed in the art. For example, S. P. Hiremath et al., Oriental Journal of Chemistry 13(2):173-176 (1997) discloses isoquinoline compounds allegedly useful as antifungal, antibacterial or anthelmintic agents.

S. P. Hiremath et al., Journal of the Indian Chemical Society 72(10):735-738 (1995) discloses isoquinolinone compounds.

S. P. Hiremath et al., Indian Journal of Heterocyclic Chemistry 3(1):37-42 (1993) discloses isoquinolinethione compounds allegedly useful as antifungal, antibacterial, oxytocic or anthelmintic agents.

S. P. Hiremath et al., Indian Journal of Chemistry, Section B 24B(12): 1235-1238 (1985) discloses indoloisoquinoline compounds.

U.S. Pat. No. 4,623,304 to Ishizumi et al. discloses indoloisoquinoline compounds allegedly having anti-tumor activity.

United Kingdom Patent No. GB 2025932 B2 by Sumitomo Chemical Co. discloses indoloisoquinoline compounds allegedly having bacteriacidal or fungicidal activity.

G. Winters et al., Farmaco. Ed. Sci. 34(6):507-517 (1979) discloses indoloisoquinolinones allegedly having antibacterial or fungicidal activity.

U.S. Pat. No. 4,113,731 to G. Winters et al. discloses indoloisoquinolines.

U.S. Pat. Nos. 5,733,918, 5,710,162, and 6,028,079 to Okazaki et al. disclose indenoquinolines allegedly useful as antitumor agents.

S. Srivastava et al., Journal of the Indian Chemical Society 66(4):276-81 (1989) discloses a synthesis of indenoisocoumarins and indenoisoquinolones.

G. Jha et al., Indian Journal of Chemistry, Section B 24B(4):440-444 (1985) discloses a synthesis of indenoisocoumarins and indenoisoquinolones.

J. N. Chatterjea et al., J. Indian Chem. Soc. 44(11):911-919 (1967) discloses a synthesis of dihydroisocoumarins.

There remains, however, a need in the art for compounds useful for treating or preventing an inflammatory disease, a reperfusion injury, an ischemic condition, renal failure, diabetes, a diabetic complication, a vascular disease, or cancer.

Citation of any reference in Section 2 of this application is not to be construed as an admission that such reference is prior art to the present application.

### 3. SUMMARY OF THE INVENTION

The present invention encompasses compounds having the Formula (I): 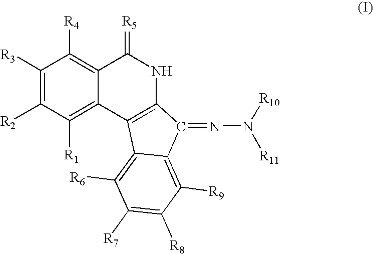
and pharmaceutically acceptable salts thereof,
wherein:
R¹, R², R³, R⁴, R⁶R⁷, R⁸ and R⁹ are independently —H, -halo, —OH, —NH₂, —CN, —NO₂, or -A-B;
R⁵ is O, S or NH;
A is —SO₂—, —SO₂NH—, —NHSO₂—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form a nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a nitrogen-containing 7- to 10-membered bicyclic heterocycle);
R¹⁰ is —H, —C₁-C₅ alkyl, —(CH₂)ₙ—CN, —(CH₂)ₙ-aryl, —(CH₂)ₙ-(3- to 7-membered monocyclic heterocycle), —(CH₂)ₙ-(7- to 10-membered bicyclic heterocycle), —(CH₂)ₙ—COO—(C₁-C₅ alkyl), —(CH₂)ₙ—COO-aryl, —(CH₂)ₙ—COOH, —CONH—(CH₂)ₙ—COOH, —CONH—(CH₂)ₙ—COO—(C₁-C₅ alkyl), —CONH—(CH₂)ₙ-aryl, —CONHNH—(C₁-C₅ alkyl), —CONHNH-aryl, —(CH₂)ₙ—CONH₂, —(CH₂)ₙ—CONH—(C₁-C₅ alkyl), —(CH₂), —CONH-aryl, —(CH₂)ₙ—CONH—(CH₂)_{q}-aryl, —(CH₂)ₙ—CONH—(CH₂)_{q}-(3- to 7-membered monocyclic heterocycle), —(CH₂)ₙ—CONH—(CH₂)_{q}-(7- to 10-membered monocyclic heterocycle), —(CH₂)ₙ—CONH—(CH₂)_{q}—CONH₂—(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)(CH₂)ₙ—(C₁-C₅ alkyl), —C(O)(CH₂)ₙ-aryl, —C(O)(CH₂)ₙ—COOH, —C(O)(CH₂)ₙ—COO—(C₁-C₅ alkyl), —C(O)(CH₂)ₙ—COO-(3- to 7-membered monocyclic heterocycle), —C(O)(CH₂), —COO-(7- to 10-membered bicyclic heterocycle), —C(O)(CH₂)ₙ-phenyl, —C(O)(CH₂)ₙ-(3- to 7-membered monocyclic heterocycle), —C(O)(CH₂)ₙ-(7- to 10-membered bicyclic heterocycle), —C(O)O(CH₂)ₙ-phenyl, —C(O)O(CH₂)ₙ-(3- to 7-membered monocyclic heterocycle), —C(O)O(CH₂)ₙ-(7- to 10-membered bicyclic heterocycle), —C(O)N((CH₂)ₙ-phenyl)₂, —C(O)N((CH₂)ₙ-phenyl)((CH₂)_{q}-3- to 7-membered monocyclic heterocycle), —C(O)N((CH₂)ₙ-phenyl)((CH₂)_{q} 7- to 10-membered bicyclic heterocycle), —C(O)N((CH₂)ₙ-(3- to 7-membered monocyclic heterocycle)₂, —C(O)N((CH₂)ₙ-7- to 10-membered bicyclic heterocycle)₂, or —SO₂NH₂;
R¹¹ is —H, or (—C₁-C₆ alkyl), or R₁₀, R₁₁ and the nitrogen atom to which they are attached join to form a -(nitrogen-containing 3- to 7-membered monocyclic heterocycle), or a -(nitrogen-containing 7- to 10-membered bicyclic heterocycle);
each n is independently an integer ranging from 0 to 10;
each p is independently an integer ranging from 0 to 5; and
each q is independently an integer ranging from 0 to 10.

The present invention also encompasses compounds having the Formula (II): 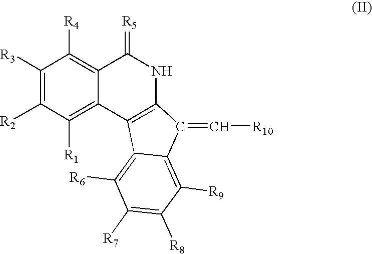
and pharmaceutically acceptable salts thereof,
wherein:
R¹, R², R³, R⁴, R⁶, R⁷, R⁸ and R⁹ are independently —H, -halo, —OH, —NH₂, —CN, —NO₂, or -A-B;
R⁵ is O, S or NH;
A is —SO₂—, —SO₂NH—, —NHSO₂—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);
R¹⁰ is —H, —C₁-C₅ alkyl, —(CH₂)ₙ—CN, —(CH₂)ₙ-aryl, —(CH₂)ₙ-(3- to 7-membered monocyclic heterocycle), —(CH₂), -(7- to 10-membered bicyclic heterocycle), —(CH₂), —COO—(C₁-C₅ alkyl), —(CH₂)ₙ—COO-aryl, —(CH₂), —COOH, —CONH—(CH₂)ₙ—COOH, —CONH—(CH₂), —COO—(C₁-C₅ alkyl), —CONH—(CH₂)ₙ-aryl, —CONHNH—(C₁-C₅ alkyl), —CONHNH-aryl, —(CH₂)ₙ—CONH₂, —(CH₂)ₙ—CONH—(C₁-C₅ alkyl), —(CH₂), —CONH-aryl, —(CH₂)ₙ—CONH—(CH₂)_{q}-aryl, —(CH₂)ₙ—CONH—(CH₂)_{q}-(3- to 7-membered monocyclic heterocycle), —(CH₂)ₙ—CONH—(CH₂)_{q}-(7- to 10-membered bicyclic heterocycle), —(CH₂)ₙ—CONH—(CH₂)_{q}—CONH₂—(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)(CH₂)ₙ—(C₁-C₅ alkyl), —C(O)(CH₂)ₙ-aryl, —C(O)(CH₂), —COOH, —C(O)(CH₂)ₙ—COO—(C₁-C₅ alkyl), —C(O)(CH₂)ₙ—COO-(3- to 7-membered monocyclic heterocycle), —C(O)(CH₂)ₙ—COO-(7- to 10-membered bicyclic heterocycle), —C(O)(CH₂)ₙ-phenyl, —C(O)(CH₂)ₙ-(3- to 7-membered monocyclic heterocycle), —C(O)(CH₂), -(7- to 10-membered bicyclic heterocycle), —C(O)O(CH₂)ₙ-phenyl, —C(O)O(CH₂)ₙ-(3- to 7-membered monocyclic heterocycle), —C(O)O(CH₂), -(7- to 10-membered bicyclic heterocycle), —C(O)N((CH₂)ₙ-phenyl)₂, —C(O)N((CH₂)ₙ-phenyl)((CH₂)_{q}-3- to 7-membered monocyclic heterocycle), —C(O)N((CH₂)ₙ-phenyl)((CH₂)_{q}-7- to 10-membered bicyclic heterocycle), —C(O)N((CH₂)ₙ-3- to 7-membered monocyclic heterocycle)₂, —C(O)N((CH₂)ₙ-7- to 10-membered bicyclic heterocycle)₂, or —SO₂NH₂;
each n is independently an integer ranging from 0 to 10;
each p is independently an integer ranging from 0 to 5; and
each q is independently an integer ranging from 0 to 10.

The present invention further encompasses compounds having the Formula (III): 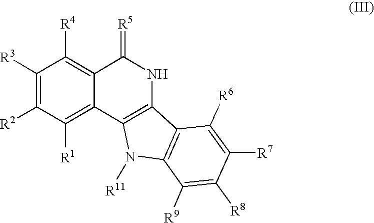
and pharmaceutically acceptable salts thereof,
wherein:
R¹, R², R³, R⁴, R⁶, R⁷, R⁸ and R⁹ are each independently —H, —O—(C₁-C₅ alkyl), —C₁-C₁₀ alkyl, —C₂-C₁₀ alkenyl, -aryl, —C(O)OH, —C(O)O(C₁-C₅ alkyl), —OC(O)(C₁-C₅ alkyl), —NO₂, —NHC(O)(CH₂)ₙ—NH₂, —NHSO₂NH(CH₂)ₙ—NH₂, —C(O)NH(CH₂)ₙ—NH₂, —SO₂NH(CH₂)ₙ—NH₂, -halo, —OH, —NH₂, or -A-B;
R⁵ is O, S or NH;
A is —SO₂—, —SO₂NH—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);
R¹¹ is —H, —C₁-C₅, alkyl, —(CH₂)ₙ-aryl, —C(O)R¹², —C(O)OR¹², —C(O)O—(C₁-C₅ alkyl), —CONH₂, —C(O)NH—(CH₂)ₙ, —C(O)OH, —(CH₂)ₙ—C(O)OH, —(CH₂)ₙ—CONH—(CH₂)_{q}-(3- to 7-membered monocyclic heterocycle), —(CH₂)ₚ-(3- to 7-membered bicyclic heterocycle), —(CH₂)ₚ-(7- to 10-membered bicyclic heterocycle), —(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)—(CH₂)ₙ—C(O)O—(C₁-C₅ alkyl), —CONH—(CH₂)ₚ(3- to 7-membered monocyclic heterocycle), —C(O)N(R¹²)₂, —C(O)NHNHR¹², —CONH(CH₂)ₙN(R¹²)₂, —CONHN(Z₁)(Z₂), or -A-B;
each occurrence of R¹² is independently —H, —(C₁-C₅ alkyl), —(CH₂)ₚ-phenyl, —(CH₂)ₚ-(3- to 7-membered monocyclic heterocycle), or —(CH₂)ₚ-(7- to 10-membered bicyclic heterocycle);
each n is independently an integer ranging from 1 to 10;
each p is independently an integer ranging from 0 to 5; and
each q is independently an integer ranging from 0 to 10.

The present invention also encompasses compounds having the Formula (IV): 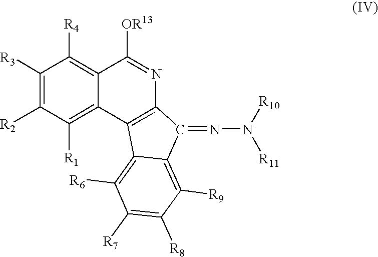
and pharmaceutically acceptable salts thereof,
wherein:
R¹, R², R³, R⁴, R⁶, R⁷, R⁸ and R⁹ are independently —H, -halo, —OH, —NH₂, —CN, —NO₂, or -A-B;
A is —SO₂—, —SO₂NH—, —NHSO₂—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);
R¹⁰ is —H, —C₁-C₅ alkyl, —(CH₂), —CN, —(CH₂)ₙ-aryl, —(CH₂)ₙ-(3- to 7-membered monocyclic heterocycle), —(CH₂)ₙ-(7- to 10-membered bicyclic heterocycle), —(CH₂)ₙ—COO—(C₁-C₅ alkyl), —(CH₂), —COO-aryl, —(CH₂)ₙ—COOH, —CONH—(CH₂)ₙ—COOH, —CONH—(CH₂)ₙ—COO—(C₁-C₅ alkyl), —CONH—(CH₂)ₙ-aryl, —CONHNH—(C₁-C₅ alkyl), —CONHNH-aryl, —(CH₂), —CONH₂, —(CH₂)ₙ—CONH—(C₁-C₅ alkyl), —(CH₂)ₙ—CONH-aryl, —(CH₂)ₙ—CONH—(CH₂)_{q}-aryl, —(CH₂)ₙ—CONH—(CH₂)_{q}-(3- to 7-membered monocyclic heterocycle), —(CH₂)ₙ—CONH—(CH₂)_{q}-(7- to 10-membered monocyclic heterocycle), —(CH₂)ₙ—CONH—(CH₂)_{q}—CONH₂—(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)(CH₂), —(C₁-C₅ alkyl), —C(O)(CH₂)ₙ-aryl, —C(O)(CH₂)ₙ—COOH, —C(O)(CH₂), —COO—(C₁-C₅ alkyl), —C(O)(CH₂)ₙ—COO-(3- to 7-membered monocyclic heterocycle), —C(O)(CH₂)ₙ—COO-(7- to 10-membered bicyclic heterocycle), —C(O)(CH₂)ₙ-phenyl, —C(O)(CH₂)ₙ-(3- to 7-membered monocyclic heterocycle), —C(O)(CH₂)ₙ-(7- to 10-membered bicyclic heterocycle), —C(O)O(CH₂)ₙ-phenyl, —C(O)O(CH₂)ₙ-(3- to 7-membered monocyclic heterocycle), —C(O)O(CH₂)ₙ-(7- to 10-membered bicyclic heterocycle), —C(O)N((CH₂)ₙ-phenyl)₂, —C(O)N((CH₂)ₙ-phenyl)((CH₂)_{q}-(3- to 7-membered monocyclic heterocycle), —C(O)N((CH₂)ₙ-phenyl)((CH₂)_{q} 7- to 10-membered bicyclic heterocycle), —C(O)N((CH₂)ₙ-(3- to 7-membered monocyclic heterocycle)₂, —C(O)N((CH₂)ₙ-7- to 10-membered bicyclic heterocycle)₂, or —SO₂NH₂;
R¹¹ is —H, or (—C₁-C₆ alkyl), or R₁₀, R₁₁ and the nitrogen atom to which they are attached join to form a -(nitrogen-containing 3- to 7-membered monocyclic heterocycle), or a -(nitrogen-containing 7- to 10-membered bicyclic heterocycle);
R¹³ is —C₁-C₁₀ alkyl, —C(O)—C₁-C₁₀ alkyl, —C(O)-aryl, —C(O)-(3- to 7-membered monocyclic heterocycle), or -glycoside, each of which is unsubstituted or substituted with one or more -halo, —C(O)OH, or —OH groups;
each n is independently an integer ranging from 0 to 10;
each p is independently an integer ranging from 0 to 5; and
each q is independently an integer ranging from 0 to 10.

The present invention also encompasses compounds having the Formula (V): 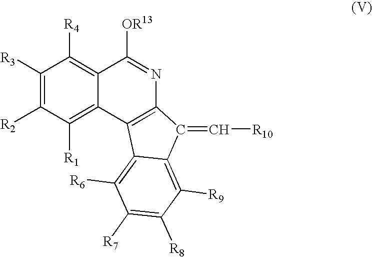
and pharmaceutically acceptable salts thereof,
wherein:
R¹, R², R³, R⁴, R⁶, R⁷, R⁸ and R⁹ are independently —H, -halo, —OH, —NH₂, —CN, —NO₂, or -A-B;
A is —SO₂—, —SO₂NH—, —NHSO₂—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);
R¹⁰ is —H, —C₁-C₅ alkyl, —(CH₂), —CN, —(CH₂)ₙ-aryl, —(CH₂)ₙ-(3- to 7-membered monocyclic heterocycle), —(CH₂)ₙ-(7- to 10-membered bicyclic heterocycle), —(CH₂)ₙ—COO—(C₁-C₅ alkyl), —(CH₂)ₙ, —COO-aryl, —(CH₂)ₙ—COOH, —CONH—(CH₂)ₙ—COOH, —CONH—(CH₂)ₙ—COO—(C₁-C₅ alkyl), —CONH—(CH₂)ₙ-aryl, —CONHNH—(C₁-C₅ alkyl), —CONHNH-aryl, —(CH₂)ₙ, —CONH₂, —(CH₂)ₙ, —CONH—(C₁-C₅ alkyl), —(CH₂)ₙ—CONH-aryl, —(CH₂)ₙ—CONH—(CH₂)_{q}-aryl, —(CH₂)ₙ—CONH(CH₂)_{q}-(3- to 7-membered monocyclic heterocycle), —(CH₂)ₙ—CONH—(CH₂)_{q}-(7- to 10-membered bicyclic heterocycle), —(CH₂)ₙ—CONH—(CH₂)_{q}—CONH₂—(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)(CH₂)ₙ—C₁-C₅ alkyl, —C(O)(CH₂)ₙ-aryl, —C(O)(CH₂)ₙ—COOH, —C(O)(CH₂)ₙ—COO—(C₁-C₅ alkyl), —C(O)(CH₂), —COO-(3- to 7-membered monocyclic heterocycle), —C(O)(CH₂), —COO-(7- to 10-membered bicyclic heterocycle), —C(O)(CH₂)ₙ-phenyl, —C(O)(CH₂)ₙ-(3- to 7-membered monocyclic heterocycle), —C(O)(CH₂)ₙ-(7- to 10-membered bicyclic heterocycle), —C(O)O(CH₂)ₙ-phenyl, —C(O)O(CH₂)ₙ-(3- to 7-membered monocyclic heterocycle), —C(O)O(CH₂)ₙ-(7- to 10-membered bicyclic heterocycle), —C(O)N((CH₂)ₙ-phenyl)₂, —C(O)N((CH₂)ₙ-phenyl)((CH₂)_{q}-3- to 7-membered monocyclic heterocycle), —C(O)N((CH₂)ₙ-phenyl)((CH₂)_{q}-7- to 10-membered bicyclic heterocycle), —C(O)N((CH₂)ₙ-3- to 7-membered monocyclic heterocycle)₂, —C(O)N((CH₂)ₙ-7- to 10-membered bicyclic heterocycle)₂, or —SO₂NH₂;
R¹³ is —C¹—C₁₀ alkyl, —C(O)—C₁-C₁₀ alkyl, —C(O)-aryl, —C(O)-(3- to 7-membered monocyclic heterocycle), or -glycoside, each of which is unsubstituted or substituted with one or more -halo, —C(O)OH, or —OH groups;
each n is independently an integer ranging from 0 to 10;
each p is independently an integer ranging from 0 to 5; and
each q is independently an integer ranging from 0 to 10.

The present invention further encompasses compounds having the Formula (VI): 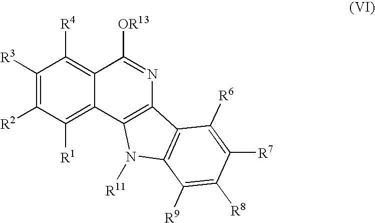
and pharmaceutically acceptable salts thereof,
wherein:
R¹, R², R³, R⁴, R⁶, R⁷, R⁸ and R⁹ are each independently —H, —O—(C₁-C₅ alkyl), —C₁-C₁₀ alkyl, —C₂-C₁₀ alkenyl, -aryl, —C(O)OH, —C(O)O(C₁-C₅ alkyl), —OC(O)(C₁-C₅ alkyl), —NO₂, —NHC(O)(CH₂)ₙ—NH₂, —NHSO₂NH(CH₂)ₙ—NH₂, —C(O)NH(CH₂)ₙ—NH₂, —SO₂NH(CH₂)ₙ—NH₂-halo, —OH, —NH₂, or -A-B;
A is —SO₂—, —SO₂NH—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);
R¹¹ is —H, —C₁-C₅ alkyl, —(CH₂)ₙ-aryl, —C(O)R¹², —C(O)OR¹², —C(O)O—(C₁-C₅ alkyl), —CONH₂, —C(O)NH—(CH₂)ₙ—C(O)OH, —(CH₂)ₙ—C(O)OH, —(CH₂)ₙ—CONH—(CH₂)_{q}-(3- to 7-membered monocyclic heterocycle), —(CH₂)ₚ-(3- to 7-membered bicyclic heterocycle), —(CH₂)ₚ-(7- to 10-membered bicyclic heterocycle), —(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)—(CH₂)ₙ—C(O)O—(C₁-C₅ alkyl), —CONH—(CH₂)ₚ-(3- to 7-membered monocyclic heterocycle), —C(O)N(R¹²)₂, —C(O)NHNHR¹², —CONH(CH₂)ₙN(R¹²)₂, —CONHN(Z₁)(Z₂), or -A-B;
R¹³ is —C₁-C₁₀ alkyl, —C(O)—C₁-C₁₀ alkyl, —C(O)-aryl, —C(O)-(3- to 7-membered monocyclic heterocycle), or -glycoside, each of which is unsubstituted or substituted with one or more -halo, —C(O)OH, or —OH groups;
each occurrence of R¹² is independently —H, —(C₁-C₅ alkyl), —(CH₂)ₚ-phenyl, —(CH₂)ₚ-(3- to 7-membered monocyclic heterocycle), or —(CH₂)ₚ-7- to 10-membered bicyclic heterocycle;
each n is independently an integer ranging from 1 to 10;
each p is independently an integer ranging from 0 to 5; and
each q is independently an integer ranging from 0 to 10.

A compound of Formula (I), (II), (III), (IV), (V), or (VI), or a pharmaceutically acceptable salt thereof (a “Tetracyclic Lactam Derivative”) is useful for treating or preventing an inflammatory disease, a reperfusion injury, an ischemic condition, renal failure, diabetes, a diabetic complication, a vascular disease, reoxygenation injury resulting from organ transplantation, Parkinson's disease, or cancer (each being a “Condition”) in an animal.

The invention also relates to compositions comprising an amount of a Tetracyclic Lactam Derivative that is effective to treat or prevent a Condition, and a physiologically acceptable carrier or vehicle. The compositions are useful for treating or preventing a Condition in an animal.

The invention further relates to methods for treating or preventing a Condition, comprising administering to an animal in need thereof an amount of a Tetracyclic Lactam Derivative that is effective to treat or prevent the Condition.

The present invention may be understood more fully by reference to the following detailed description and illustrative examples, which are intended to exemplify non-limiting embodiments of the invention.

### 4. DETAILED DESCRIPTION OF THE INVENTION

### 4.1 Tetracyclic Lactam Derivatives of Formula (I)

As stated above, the present invention encompasses Tetracyclic Lactam Derivatives of Formula (I) 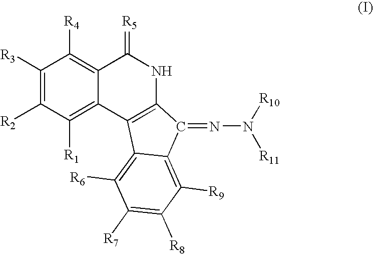
where R¹, R², R³, R⁴, R⁵, R⁶, R⁷, R⁸, R⁹, R¹⁰ and R¹¹ are defined above for the Tetracyclic Lactam Derivatives of Formula (I).

In one embodiment R¹, R², R³ and R⁴ are independently —H, —NO₂, —NH₂, —F, —OH, or —O—(C₁-C₅ alkyl).

In another embodiment R¹, R², R³ and R⁴ are each —H.

In another embodiment R², R³ and R⁴ are each —H.

In another embodiment R⁶, R⁷ and R⁹ are each —H.

In another embodiment R¹, R², R³, R⁴, R⁶, R⁷, R⁸ and R⁹ are each —H.

In another embodiment R⁵ is oxygen.

In still another embodiment R¹, R², R³ and R⁴ are each hydrogen.

In yet another embodiment R⁶, R⁷, R⁸ or R⁹ is -A-B, where A is —NHC(O)— and B is —(C₁-C₅ alkylene)-NZ₁Z₂.

In a further embodiment R⁶, R⁷, R⁸ or R⁹ is -A-B, where A is —SO₂NH—; B is —C₁-C₅ alkylene)-N(Z₁)(Z₂); and N, Z₁ and Z₂ are taken together to form a nitrogen-containing 3- to 7-membered monocyclic heterocycle.

In another embodiment R⁸ is —NHC(O)CH₂N(CH₃)₂.

In a further embodiment R⁸ is —SO₂NH(CH₂)₃-(morpholin-4-yl).

In one embodiment R¹⁰ is —H, —C₁-C₅ alkyl, —(CH₂)ₙ-aryl, —COO—(C₁-C₅ alkyl), —CONH₂, —CONH—(CH₂)ₙ—COOH, —(CH₂)ₙ—CONH—(CH₂)_{q}-(3- to 7-membered monocyclic heterocycle), —(CH₂)ₙ—CONH—(CH₂)_{q}-(7- to 10-membered bicyclic heterocycle), —(CH₂), —CONH—(CH₂)_{q}—CONH—(C₁-C₅ alkyl), —(CH₂)ₙ—CONH—(CH₂)_{q}—CON—(C₁-C₅ alkyl)₂, —C(O)—(C₁-C₅ alkyl) or —(C(O)(CH₂)ₙ—COO—(C₁-C₅ alkyl).

In another embodiment R⁵ is NH.

In yet another embodiment R⁵ is S.

In one embodiment, the Tetracyclic Lactam Derivatives of Formula (I) are in isolated and purified form.

In another embodiment, the Tetracyclic Lactam Derivatives of Formula (I) have the formula (Ia): 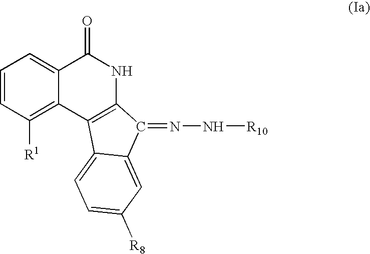
where R₁, R₈ and R¹⁰ are as defined above for the Tetracyclic Lactam Derivatives of Formula (I).

Illustrative examples of the compounds of Formula (Ia) are as set forth below.

| Compound | R¹ | R⁸ | R¹⁰ |
|---|---|---|---|
| 1 | —H | —H | H |
| 2 | —H | —H | —CH₃ |
| 3 | —H | —H | —CH₂CH₃ |
| 4 | —H | —H | -benzyl |
| 5 | —H | —H | —COOCH₃ |
| 6 | —H | —H | —COCH₂COOCH₃ |
| 7 | —H | —H | —CH₂CH₂CN |
| 8 | —H | —H | —COCH₃ |
| 9 | —H | —H | —CONHCH₃ |
| 10 | —H | —H | —CH₂CH₂-(tetrazol-5-yl) |
| 11 | —H | —H | —CONH(CH₂)₂N(CH₃)₂ |
| 12 | —H | —H | —CONH(CH₂)₂-(morpholin-4-yl) |
| 13 | —H | —H | —CONH(CH₂)₃-(morpholin-4-yl) |
| 14 | —H | —H | —CONH(CH₂)₂COOCH₂CH₃ |
| 15 | —H | —H | —CONH(CH₂)₂COOH |
| 16 | —H | —H | —CONH(CH₂)₂CONHCH₃ |
| 17 | —H | —H | —CONH-(piperidin-1-yl) |
| 18 | —H | —H | —CONH-(morpholin-4-yl) |
| 19 | —H | —H | —CO(CH₂)₂-(tetrazol-5-yl) |
| 20 | —H | —NHC(O)CH₂N(CH₃)₂ | —H |
| 21 | —H | —SO₂NH(CH₂)₃-(morpholin-4-yl) | —H |
| 22 | —H | —NHC(O)CH₂N(CH₃)₂ | —COCH₃ |
| 23 | —H | —SO₂NH(CH₂)₃-(morpholin-4-yl) | —COCH₃ |
| 24 | —H | —NHC(O)CH₂N(CH₃)₂ | —CONHCH₃ |
| 25 | —H | —SO₂NH(CH₂)₃-(morpholin-4-yl) | —CONHCH₃ |
| 26 | —NH₂ | —NHC(O)CH₂N(CH₃)₂ | —CONH₂ |
| 27 | —OH | —SO₂NH(CH₂)₃-(morpholin-4-yl) | —CONH₂ |
| 28 | —F | —NHC(O)CH₂N(CH₃)₂ | —CONHCH₃ |
| 29 | —OCH₃ | —SO₂NH(CH₂)₃-(morpholin-4-yl) | —CONHCH₃ |
| 112 | —H | —H | —(CH₂)₂-(tetrazol-5-yl) |

and pharmaceutically acceptable salts thereof.

In another embodiment, the Tetracyclic Lactam Derivatives of Formula (I) have the formula (Ib): 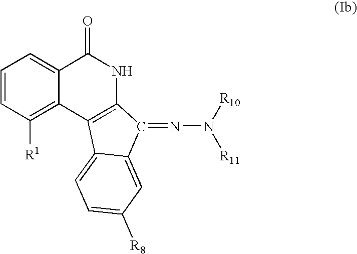
where R₁₀, R₁₁ and the nitrogen atom to which they are attached join to form a -(nitrogen-containing 3- to 7-membered monocyclic heterocycle);
and wherein R₁, R₈ are as defined above for the Tetracyclic Lactam Derivatives of Formula (I).

Illustrative examples of the compounds of Formula (Ib) are as set forth below.

| Compound | R¹ | R⁸ | —NR¹⁰R¹¹ |
|---|---|---|---|
| 105 | —H | —H | -(morpholin-4-yl) |
| 106 | —H | —H | -(4-methyl-piperazin-1-yl) |

and pharmaceutically acceptable salts thereof.

### 4.2 Tetracyclic Lactam Derivatives of Formula (II)

As stated above, the present invention encompasses Tetracyclic Lactam Derivatives of Formula (II): 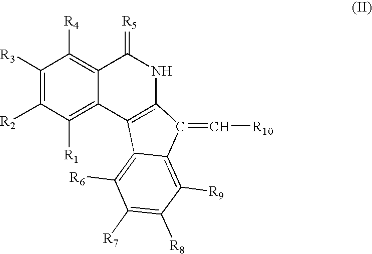
where R¹, R², R³, R⁴, R⁵, R⁶, R⁷, R⁸, R⁹ and R¹⁰ are defined above for the Tetracyclic Lactam Derivatives of Formula (II).

In one embodiment R¹, R², R³ and R⁴ are independently —H, —F, —NO₂, —NH₂, —OH, or —O—(C₁-C₅ alkyl).

In another embodiment R¹, R², R³ and R⁴ are each —H.

In another embodiment R², R³ and R⁴ are each —H.

In another embodiment R⁶, R⁷ and R⁹ are each —H.

In another embodiment R¹, R², R³, R⁴, R⁶, R⁷, R⁸ and R⁹ are each hydrogen.

In another embodiment R⁵ is oxygen.

In yet another embodiment R⁶, R⁷, R⁸ or R⁹ is -A-B, where A is —NHC(O)— and B is —(C₁-C₅ alkylene)-NZ₁Z₂.

In a further embodiment R⁶, R⁷, R⁸ or R⁹ is -A-B, where A is —SO₂NH— and B is —C₁-C₁₀ alkyl, wherein the —C₁-C₁₀ alkyl group is substituted with a heterocyclic amine.

In another embodiment R⁸ is —NHC(O)CH₂N(CH₃)₂.

In a further embodiment R⁸ is —SO₂NH(CH₂)₃-(morpholin-4-yl).

In one embodiment R¹⁰ is —H, —C₁-C₅ alkyl, —(CH₂)ₙ-aryl, —COO—(C₁-C₅ alkyl), —CONH₂, —(CH₂)ₙ-(3- to 7-membered monocyclic heterocycle), —(CH₂), -(7- to 10-membered bicyclic heterocycle), —CONH—(CH₂), —COOH, —(CH₂)ₙ—CONH—(CH₂)_{q}-(3- to 7-membered monocyclic heterocycle), —(CH₂)ₙ—CONH—(CH₂)_{q}-(7- to 10-membered bicyclic heterocycle), —(CH₂)ₙ—CONH—(CH₂)_{q}—CONH(C₁-C₅ alkyl), —(CH₂)ₙ—CONH—(CH₂)_{q}—CON—(C₁-C₅ alkyl)₂, —C(O)—(C₁-C₅) alkyl) or —C(O)(CH₂)ₙ—COO—(C₁-C₅ alkyl).

In another embodiment R⁵ is NH.

In yet another embodiment R⁵ is S.

In one embodiment, the compounds of Formula (II) are in isolated and purified form.

In another embodiment, the Tetracyclic Lactam Derivatives of Formula (II) have the formula (IIa): 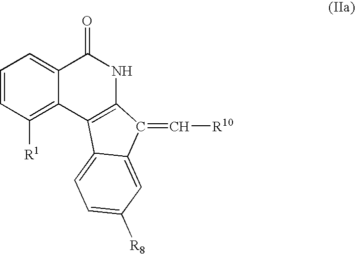
where R¹, R⁸ and R¹⁰ are defined above for the Tetracyclic Lactam Derivatives of Formula (II).

Illustrative examples of the compounds of Formula (IIa) are as set forth below.

| Compound | R¹ | R⁸ | R¹⁰ |
|---|---|---|---|
| 30 | —H | —H | —CH₃ |
| 31 | —H | —H | —CH₂CH₃ |
| 32 | —H | —H | -benzyl |
| 33 | —H | —H | —COOCH₃ |
| 34 | —H | —H | —COCH₂COOCH₃ |
| 35 | —H | —H | —COCH₂COOH |
| 36 | —H | —H | —COCH₃ |
| 37 | —H | —H | —CONH(CH₂)₂N(CH₃)₂ |
| 38 | —H | —H | —CONH(CH₂)₂-(morpholin-4-yl) |
| 39 | —H | —H | —CONH(CH₂)₃-(morpholin-4-yl) |
| 40 | —H | —H | —CONH(CH₂)₂COOCH₂CH₃ |
| 41 | —H | —H | —CONH(CH₂)₂COOH |
| 42 | —H | —H | —CONH(CH₂)₂CONHCH₃ |
| 43 | —H | —H | —CONH-(piperidin-1-yl) |
| 44 | —H | —H | —CONH-(morpholin-4-yl) |
| 45 | —H | —H | —CO(CH₂)₂-(tetrazol-5-yl) |
| 46 | —H | —NHC(O)CH₂N(CH₃)₂ | —COOCH₂CH₃ |
| 47 | —H | —SO₂NH(CH₂)₃-(morpholin-4-yl) | —COOCH₂CH₃ |
| 48 | —H | —NHC(O)CH₂N(CH₃)₂ | —COOH |
| 49 | —H | —SO₂NH(CH₂)₃-(morpholin-4-yl) | —COOH |
| 50 | —H | —NHC(O)CH₂N(CH₃)₂ | —CONHCH₃ |
| 51 | —H | —SO₂NH(CH₂)₃-(morpholin-4-yl) | —CONHCH₃ |
| 52 | —NH₂ | —NHC(O)CH₂N(CH₃)₂ | —CONH(CH₂)₂-(morpholin-4-yl) |
| 53 | —OH | —SO₂NH(CH₂)₃-(morpholin-4-yl) | —CONH(CH₂)₂N(CH₃)₂ |
| 54 | —F | —NHC(O)CH₂N(CH₃)₂ | —CONH(CH₂)₂-(morpholin-4-yl) |
| 55 | —OCH₃ | —SO₂NH(CH₂)₃-(morpholin-4-yl) | —CONH(CH₂)₂N(CH₃)₂ |

and pharmaceutically acceptable salts thereof.

### 4.3 Tetracyclic Lactam Derivatives of Formula (III)

As stated above, the present invention encompasses Tetracyclic Lactam Derivatives of Formula (I). 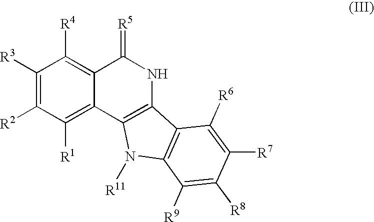
where R¹, R², R³, R⁴, R⁵, R⁷, R⁸, R⁹, and R¹¹ are defined above for the Tetracyclic Lactam Derivatives of Formula (III).

In one embodiment R¹, R², R³ and R⁴ are independently —H, —F, —NO₂, —NH₂, —OH, or —O—(C₁-C₅ alkyl).

In another embodiment R¹, R², R³ and R⁴ are each —H.

In yet another embodiment R², R³ and R⁴ are each H.

In another embodiment R⁶ and R⁹ are each —H.

In another embodiment R⁶, R⁷, R⁸ and R⁹ are each —H.

In still another embodiment R¹, R², R³, R⁴, R⁶, R⁷, R⁸ and R⁹ are each —H.

In one embodiment R⁵ is O.

In another embodiment, R⁵ is S.

In yet another embodiment, R⁵ is NH.

In another embodiment R⁷ is —H and R⁸ is -A-B, where A is —NHC(O)— and B is —(C₁-C₅ alkylene)-NZ₁Z₂.

In still another embodiment R⁸ is —H and R⁷ is -A-B, where A is —NHC(O)— and B is —(C₁-C₅ alkylene)-NZ₁Z₂.

In yet another embodiment R⁷ is —H and R⁸ is -A-B, where A is —SO₂NH—; B is —C₁-C₅ alkylene)-N(Z₁)(Z₂); and N, Z₁ and Z₂ are taken together to form a nitrogen-containing 3- to 7-membered monocyclic heterocycle.

In a further embodiment R⁸ is —H and R⁷ is -A-B, where A is —SO₂NH—; B is —C₁-C₅ alkylene)-N(Z₁)(Z₂); and N, Z₁ and Z₂ are taken together to form a nitrogen-containing 3- to 7-membered monocyclic heterocycle.

In another embodiment R⁷ is —H and R⁸ is —NHC(O)CH₂N(CH₃)₂.

In another embodiment R⁷ is —H and R⁸ is —SO₂NH(CH₂)₃-(morpholin-4-yl).

In a further embodiment R⁸ is —H and R⁷ is —SO₂NH(CH₂)₃-(morpholin-4-yl).

In one embodiment R¹¹ is —C(O)R¹², —C(O)OR¹², —C(O)NH—(CH₂)ₙ-(3- to 7-membered monocyclic heterocycle), —C(O)N(R¹²)₂, —C(O)NH(CH₂)ₙN(R¹²)₂, —C(O)NHNHR¹², —C(O)NH—N(Z₁)(Z₂), —(C₁-C₅ alkyl), —(CH₂)ₚ-phenyl, —(CH₂)ₚ-(3- to 7-membered monocyclic heterocycle), —(CH₂)ₚ-7- to 10-membered bicyclic heterocycle, or -A-B.

In another embodiment R¹¹ is —C(O)O—(C₁-C₅ alkyl), or —C(O)O—(C₁-C₅ alkyl)-NZ₁Z₂.

In a further embodiment R¹-R⁴ are each —H, R⁵ is O, and R¹¹ is —C(O)O—(C₁-C₅ alkyl), or —C(O)O—(C₁-C₅ alkyl)-NZ₁Z₂.

In one embodiment, when R¹¹ is —H and R⁵ is O, then R¹-R⁴ and R⁶-R⁹ are not simultaneously —H.

In one embodiment, the Tetracyclic Lactam Derivatives of Formula (III) are in isolated and purified form.

In another embodiment, the Tetracyclic Lactam Derivatives of Formula (III) have the formula (IIIa): 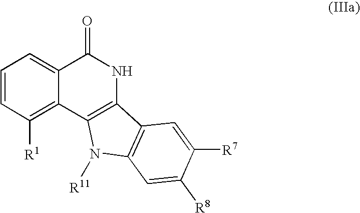
where R¹, R⁷, R⁸ and R¹¹ are as defined above for the Tetracyclic Lactam Derivatives of Formula (III).

Illustrative examples of the compounds of Formula (IIIa) are as set forth below.

| Compound | R¹ | R⁷, R⁸ | R¹¹ |
|---|---|---|---|
| 56 | —H | R⁷ = R⁸ = —H | —CH₃ |
| 57 | —H | R⁷ = R⁸ = —H | —CH₂COOCH₂CH₃ |
| 58 | —H | R⁷ = R⁸ = —H | —CH₂COOH |
| 59 | —H | R⁷ = R⁸ = —H | —CH₂CONHCH₃ |
| 60 | —H | R⁷ = R⁸ = —H | -benzyl |
| 61 | —H | R⁷ = R⁸ = —H | —COOCH₃ |
| 62 | —H | R⁷ = R⁸ = —H | —COO-t-butyl |
| 63 | —H | R⁷ = R⁸ = —H | —COOCH₂CH₃ |
| 64 | —H | R⁷ = R⁸ = —H | —COCH₃ |
| 65 | —H | R⁷ = R⁸ = —H | —CONHCH₃ |
| 66 | —H | R⁷ = R⁸ = —H | —CONHCH₂CH₃ |
| 67 | —H | R⁷ = R⁸ = —H | —CONH(CH₂)₂N(CH₃)₂ |
| 68 | —H | R⁷ = R⁸ = —H | —CONH(CH₂)₂-(morpholin-4-yl) |
| 69 | —H | R⁷ = R⁸ = —H | —CONH(CH₂)₃-(morpholin-4-yl) |
| 70 | —H | R⁷ = R⁸ = —H | —CONH(CH₂)₂COOCH₂CH₃ |
| 71 | —H | R⁷ = R⁸ = —H | —CONH(CH₂)₂COOH |
| 72 | —H | R⁷ = R⁸ = —H | —CONH(CH₂)₂CONHCH₃ |
| 73 | —H | R⁷ = R⁸ = —H | —CO(CH₂)₂-(tetrazol-5-yl) |
| 74 | —H | R⁷ = R⁸ = —H | —CONH-(piperidin-1-yl) |
| 75 | —H | R⁷ = R⁸ = —H | —CONH-(morpholin-4-yl) |
| 76 | —H | R⁷ = R⁸ = —H | —CO(CH₂)₂-(tetrazol-5-yl) |
| 77 | —H | R⁷ = R⁸ = —H | -isobutyl |
| 78 | —H | R⁷ = —NHC(O)CH₂N(CH₃)₂ | —COCH₃ |
| | | R⁸ = —H | |
| 79 | —H | R⁷ = —SO₂NH(CH₂)₃-(morpholin-4-yl) | —COCH₃ |
| | | R⁸ = —H | |
| 80 | —H | R⁷ = —NHC(O)CH₂N(CH₃)₂ | —CONHCH₃ |
| | | R⁸ = —H | |
| 81 | —H | R⁷ = —SO₂NH(CH₂)₃-(morpholin-4-yl) | —CONHCH₃ |
| | | R⁸ = —H | |
| 82 | —NH₂ | R⁷ = —NHC(O)CH₂N(CH₃)₂ | —CONHCH₃ |
| | | R⁸ = —H | |
| 83 | —OH | R⁷ = —SO₂NH(CH₂)₃-(morpholin-4-yl) | —CONHCH₃ |
| | | R⁸ = —H | |
| 84 | —F | R⁷ = —NHC(O)CH₂N(CH₃)₂ | —CONHCH₃ |
| | | R⁸ = —H | |
| 85 | —OCH₃ | R⁷ = —SO₂NH(CH₂)₃-(morpholin-4-yl) | —CONHCH₃ |
| | | R⁸ = —H | |
| 86 | —H | R⁷ = —H | —COOCH₃ |
| | | R⁸ = —NHC(O)CH₂N(CH₃)₂ | |
| 87 | —H | R⁷ = —H | —COOCH₃ |
| | | R⁸ = —SO₂NH(CH₂)₃-(morpholin-4-yl) | |
| 88 | —H | R⁷ = —H | —CONHCH₃ |
| | | R⁸ = —NHC(O)CH₂N(CH₃)₂ | |
| 89 | —H | R⁷ = —H | —CONHCH₃ |
| | | R⁸ = —SO₂NH(CH₂)₃-(morpholin-4-yl) | |
| 90 | —NH₂ | R⁷ = —H | —CONHCH₃ |
| | | R⁸ = —NHC(O)CH₂N(CH₃)₂ | |
| 91 | —OH | R⁷ = —H | —CONHCH₃ |
| | | R⁸ = —SO₂NH(CH₂)₃-(morpholin-4-yl) | |
| 92 | —F | R⁷ = —H | —CONHCH₃ |
| | | R⁸ = —NHC(O)CH₂N(CH₃)₂ | |
| 93 | —OCH₃ | R⁷ = —H | —CONHCH₃ |
| | | R⁸ = —SO₂NH(CH₂)₃-(morpholin-4-yl) | |
| 94 | —H | R⁷ = R⁸ = —H | —CON(CH₃)₂ |
| 95 | —H | R⁷ = R⁸ = —H | —CONH-(piperidin-1-yl) |
| 96 | —H | R⁷ = R⁸ = —H | —CONH-(piperidin-1-yl) |
| 97 | —H | R⁷ = R⁸ = —H | —CONH-(morpholin-1-yl) |
| 98 | —H | R⁷ = R⁸ = —H | —CONH-(4—CH₃-piperazin-1-yl) |
| 99 | —H | R⁷ = R⁸ = —H | —SO₂CH₃ |
| 100 | —H | R⁷ = R⁸ = —H | —SO₂-phenyl |
| 101 | —H | R⁷ = R⁸ = —H | —SO₃H |
| 107 | —H | R⁷ = R⁸ = —H | —C(O)O(CH₂)₂CH₃ |
| 108 | —H | R⁷ = R⁸ = —H | —C(O)OCH(CH₃)₂ |
| 109 | —H | R⁷ = R⁸ = —H | —C(O)O(CH₂)₃CH₃ |
| 110 | —H | R⁷ = R⁸ = —H | —C(O)OCH(CH₃)(CH₂CH₃) |
| 113 | —H | R⁷ = —H | —CO₂CH₂CH₃ |
| | | R⁸ = —SO₂NHN(CH₃)₂ | |
| 114 | —H | R⁷ = —SO₂NHN(CH₃)₂ | —CO₂CH₂CH₃ |
| | | R⁸ = —H | |
| 115 | —H | R⁷ = —H | —H |
| | | R⁸ = —SO₂NH(CH₂)₃-morpholin-4-yl | |
| 116 | —H | R⁷ = —SO₂NH(CH₂)₃-morpholin-4-yl | —H |
| | | R⁸ = —H | |
| 117 | —H | R⁷ = —H | —H |
| | | R⁸ = —SO₂NHN(CH₃)₂ | |
| 118 | —H | R⁷ = —SO₂NHN(CH₃)₂ | —H |
| | | R⁸ = —H | |

and pharmaceutically acceptable salts thereof.

In another embodiment, the compounds of Formula (IIa) are those wherein R¹, R⁷ and R⁸ are —H.

In yet another embodiment, the compounds of Formula (IIa) are those wherein R¹, R⁷ and R⁸ are —H; and R¹¹ is (O)O(C₁-C₅ alkyl), or —C(O)O—(C₁-C₅ alkyl)-NZ₁Z₂.

### 4.4 Tetracyclic Lactam Derivatives of Formula (IV)

As stated above, the present invention encompasses Tetracyclic Lactam Derivatives of Formula (IV) 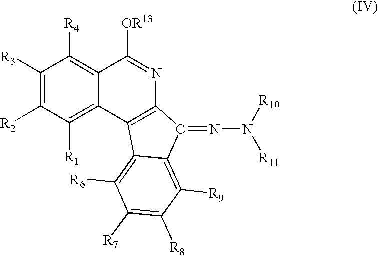
where R¹, R², R³, R⁴, R⁶, R⁷, R⁸, R⁹, R¹⁰, R¹¹ and R¹³ are defined above for the Tetracyclic Lactam Derivatives of Formula (IV).

In one embodiment R¹, R², R³ and R⁴ are independently —H, —NO₂, —NH₂, —F, —OH, or —O—(C₁-C₅ alkyl).

In another embodiment R¹, R², R³ and R⁴ are each —H.

In another embodiment R², R³ and R⁴ are each —H.

In another embodiment R⁶, R⁷ and R⁹ are each H.

In another embodiment R₁, R², R³, R⁴, R⁶, R⁷, R⁸ and R⁹ are each —H.

In still another embodiment R¹, R², R³ and R⁴ are each hydrogen.

In yet another embodiment R⁶, R⁷, R⁸ or R⁹ is -A-B, where A is —NHC(O)— and B is —(C₁-C₅ alkylene)-NZ₁Z₂.

In a further embodiment R⁶, R⁷, R⁸ or R⁹ is -A-B, where A is —SO₂NH—; B is —C₁-C₅ alkylene)-N(Z₁)(Z₂); and N, Z₁ and Z₂ are taken together to form a nitrogen-containing 3- to 7-membered monocyclic heterocycle.

In another embodiment R⁸ is —NHC(O)CH₂N(CH₃)₂.

In a further embodiment R⁸ is —SO₂NH(CH₂)₃-(morpholin-4-yl).

In one embodiment R¹⁰ is —H, —C₁-C₅ alkyl, —(CH₂)ₙ-aryl, —COO—(C₁-C₅ alkyl), —CONH₂, —CONH—(CH₂)ₙ—COOH, —(CH₂)ₙ—CONH—(CH₂)_{q}-(3- to 7-membered monocyclic heterocycle), —(CH₂)ₙ—CONH—(CH₂)_{q}-(7- to 10-membered bicyclic heterocycle), —(CH₂)ₙ—CONH—(CH₂)_{q}—CONH—(C₁-C₅ alkyl), —(CH₂)ₙ—CONH(CH₂)_{q}—CON(C₁-C₅ alkyl)₂, —C(O)—(C₁-C₅ alkyl) or —C(O)(CH₂), —COO—(C₁-C₅ alkyl).

In one embodiment, the Tetracyclic Lactam Derivatives of Formula (IV) are in isolated and purified form.

In another embodiment, the Tetracyclic Lactam Derivatives of Formula (IV) have the formula (IVa): 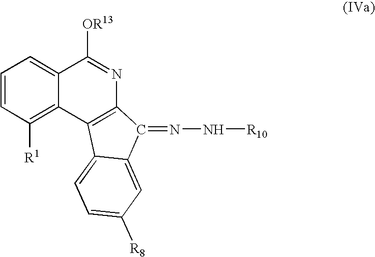
where R¹, R⁸, R¹⁰ and R¹³ are as defined above for the Tetracyclic Lactam Derivatives of Formula (IV).

In another embodiment, the Tetracyclic Lactam Derivatives of Formula (IV) have the formula (IVb): 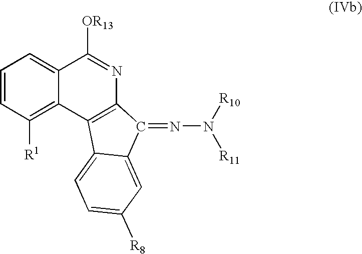
where R₁₀, R₁₁ and the nitrogen atom to which they are attached join to form a -(nitrogen-containing 3- to 7-membered monocyclic heterocycle);
and wherein R₁, R₈, and R₁₃ are as defined above for the Tetracyclic Lactam Derivatives of Formula (IV).

### 4.5 Tetracyclic Lactam Derivatives of Formula (V)

As stated above, the present invention encompasses Tetracyclic Lactam Derivatives of Formula (V): 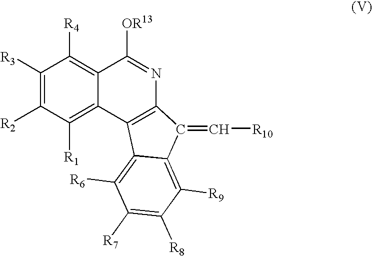
where R¹, R², R³, R⁴, R⁶, R⁷, R⁸, R⁹, R¹⁰, and R¹³ are defined above for the Tetracyclic Lactam Derivatives of Formula (V).

In one embodiment R¹, R², R³ and R⁴ are independently —H, —F, —NO₂, —NH₂, —OH, or —O—(C₁-C₅ alkyl).

In another embodiment R¹, R², R³ and R⁴ are each —H.

In another embodiment R², R³ and R⁴ are each —H.

In another embodiment R⁶, R⁷ and R⁹ are each —H.

In another embodiment R¹, R², R³, R⁴, R⁶, R⁷, R⁸ and R⁹ are each hydrogen.

In yet another embodiment R⁶, R⁷, R⁸ or R⁹ is -A-B, where A is —NHC(O)— and B is —(C₁-C₅ alkylene)-NZ₁Z₂.

In a further embodiment R⁶, R⁷, R⁸ or R⁹ is -A-B, where A is —SO₂NH— and B is —C₁-C₁₀ alkyl, wherein the —C₁-C₁₀ alkyl group is substituted with a heterocyclic amine.

In another embodiment R⁸ is —NHC(O)CH₂N(CH₃)₂.

In a further embodiment R⁸ is —SO₂NH(CH₂)₃-(morpholin-4-yl).

In one embodiment R¹⁰ is —H, —C₁-C₅ alkyl, —(CH₂)ₙ-aryl, —COO—(C₁-C₅ alkyl), —CONH₂, —(CH₂)ₙ-(3- to 7-membered monocyclic heterocycle), —(CH₂)ₙ-(7- to 10-membered bicyclic heterocycle), —CONH—(CH₂), —COOH, —(CH₂)ₙ—CONH—(CH₂)_{q}-(3- to 7-membered monocyclic heterocycle), —(CH₂)ₙ—CONH—(CH₂)_{q}-(7- to 10-membered bicyclic heterocycle), —(CH₂)ₙ—CONH(CH₂)_{q}—CONH—(C₁-C₅ alkyl), —(CH₂)ₙ—CONH—(CH₂)_{q}—CON—(C₁-C₅ alkyl)₂, —C(O) —(C₁-C₅ alkyl) or —C(O)(CH₂)ₙ—COO—(C₁-C₅ alkyl).

In one embodiment, the compounds of Formula (V) are in isolated and purified form.

In another embodiment, the Tetracyclic Lactam Derivatives of Formula (V) have the formula (Va): 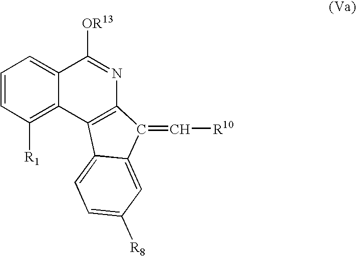
where R¹, R⁸, R¹⁰ and R¹³ are defined above for the Tetracyclic Lactam Derivatives of Formula (V).

### 4.6 Tetracyclic Lactam Derivatives of Formula (VI)

As stated above, the present invention encompasses Tetracyclic Lactam Derivatives of Formula (VI). 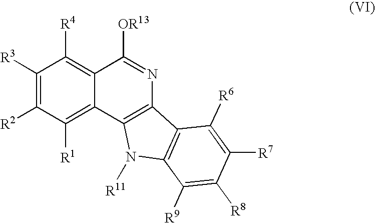
where R¹, R², R³, R⁴, R⁷, R⁸, R⁹, R¹¹, and R¹³ are defined above for the Tetracyclic Lactam Derivatives of Formula (VI).

In one embodiment R¹, R², R³ and R⁴ are independently —H, —F, —NO₂, —NH₂, —OH, or —O—(C₁-C₅ alkyl).

In another embodiment R¹, R², R³ and R⁴ are each —H.

In yet another embodiment R², R³ and R⁴ are each —H.

In another embodiment R⁶ and R⁹ are each —H.

In another embodiment R⁶, R⁷, R⁸ and R⁹ are each —H.

In still another embodiment R¹, R², R³, R⁴, R⁶, R⁷, R⁸ and R⁹ are each —H.

In another embodiment R⁷ is —H and R⁸ is -A-B, where A is —NHC(O)— and B is —(C₁-C₅ alkylene)-NZ₁Z₂.

In still another embodiment R⁸ is —H and R⁷ is -A-B, where A is —NHC(O)— and B is —(C₁-C₅ alkylene)-NZ₁Z₂.

In yet another embodiment R⁷ is —H and R⁸ is -A-B, where A is —SO₂NH—; B is —C₁-C₅ alkylene)-N(Z₁)(Z₂); and N, Z₁ and Z₂ are taken together to form a nitrogen-containing 3- to 7-membered monocyclic heterocycle.

In a further embodiment R⁸ is —H and R⁷ is -A-B, where A is —SO₂NH—; B is —C C₅ alkylene)-N(Z₁)(Z₂); and N, Z₁ and Z₂ are taken together to form a nitrogen-containing 3- to 7-membered monocyclic heterocycle.

In another embodiment R⁷ is —H and R⁸ is —NHC(O)CH₂N(CH₃)₂.

In another embodiment R⁷ is —H and R⁸ is —SO₂NH(CH₂)₃-(morpholin-4-yl).

In a further embodiment R⁸ is —H and R⁷ is —SO₂NH(CH₂)₃-(morpholin-4-yl).

In one embodiment R¹¹ is —C(O)R¹², —C(O)OR¹², —C(O)NH—(CH₂)ₚ-(3- to 7-membered monocyclic heterocycle), —C(O)N(R¹²)₂, —C(O)NH(CH₂)ₙN(R¹²)₂, —C(O)NHNHR¹², —C(O)NH—N(Z₁)(Z₂), —(C₁-C₅ alkyl), —(CH₂)ₚ-phenyl, —(CH₂)ₚ-(3- to 7-membered monocyclic heterocycle), —(CH₂)ₚ-7- to 10-membered bicyclic heterocycle, or -A-B.

In another embodiment R¹¹ is —C(O)O—(C₁-C₅ alkyl), or —C(O)O—(C₁-C₅ alkyl)-NZ₁Z₂.

In a further embodiment R¹-R⁴ are each —H, and R¹¹ is —C(O)O—(C₁-C₅ alkyl), or —C(O)O—(C₁-C₅ alkyl)-NZ₁Z₂.

In another embodiment, the compounds of Formula (VIa) are those wherein R¹, R⁷ and R⁸ are —H.

In yet another embodiment, the compounds of Formula (VIa) are those wherein R¹, R⁷ and R⁸ are —H; and R¹¹ is —C(O)O(C₁-C₅ alkyl), or —C(O)O—(C₁-C₅ alkyl)-NZ₁Z₂.

In one embodiment, when R¹¹ is —H and R⁵ is O, then R¹-R⁴ and R⁶-R⁹ are not simultaneously —H.

In one embodiment, the Tetracyclic Lactam Derivatives of Formula (VI) are in isolated and purified form.

In another embodiment, the Tetracyclic Lactam Derivatives of Formula (VI) have the formula (VIa): 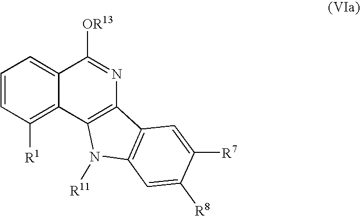
where R¹, R⁷, R⁸, R¹¹ and R¹³ are as defined above for the Tetracyclic Lactam Derivatives of Formula (VI).

### 4.7 Tetracyclic Lactam Derivatives of Formulas (I), (II), and (III)

The Tetracyclic Lactam Derivatives can exist in a keto or enol tautomeric form. This invention encompasses both the keto and enol forms of the Tetracyclic Lactam Derivatives. Accordingly, Formulas (I), (II), and (III), although depicting the keto form of the Tetracyclic Lactam Derivatives, encompass both the keto and enol forms.

The present invention also includes Tetracyclic Lactam Derivatives, wherein one or more hydrogen, carbon or other atoms are replaced by an isotope thereof. Such compounds are useful as research or diagnostic tools in metabolism pharmacokinetic studies and in binding assays.

### 4.8 Definitions

As used herein, the terms used above having following meaning:

The term “—(C₁-C₁₀)alkyl” as used herein, refers to a straight chain or branched non-cyclic hydrocarbon having from 1 to 10 carbon atoms. Representative straight chain —(C₁-C₁₀)alkyls include -methyl, -ethyl, -n-propyl, -n-butyl, -n-pentyl, -n-hexyl, -n-heptyl, -n-octyl, -n-nonly and -n-decyl. Representative branched —(C₁-C₁₀)alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl and 3,3-dimethylbuty, -isopropyl, -sec-butyl, -isobutyl, 1-methylhexyl, 2-methylhexyl, 3-methylhexyl, 4-methylhexyl, 5-methylhexyl, 1,2-dimethylpentyl, 1,3-dimethylpentyl, 1,2-dimethylhexyl, 1,3-dimethylhexyl, 3,3-dimethylhexyl, 1,2-dimethylheptyl, 1,3-dimethylheptyl, and 3,3-dimethylheptyl.

The term “—(C₁-C₅)alkyl” as used herein, refers to a straight chain or branched non-cyclic hydrocarbon having from 1 to 5 carbon atoms. Representative straight chain —(C₁-C₅)alkyls include -methyl, -ethyl, -n-propyl, -n-butyl and -n-pentyl. Representative branched —(C₁-C₅)alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl and 1,2-dimethylpropyl. Representative examples of a C₁-C₅ alkyl substituted with a halo group include, but are not limited to —CH₂F, —CCl₃, —CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃ and —C(CH₃)₂(CH₂Cl). Representative examples of a C₁-C₅ alkyl substituted with an —NH₂ group include, but are not limited to —CH₂NH₂, —CH₂CH₂NH₂, —CH₂CH₂CH₂NH₂, —CH₂CH₂CH₂CH₂NH₂, —CH₂CH(NH₂)CH₃, —CH₂CH(NH₂)CH₂CH₃, —CH(NH₂)CH₂CH₃, —C(CH₃)₂(CH₂NH₂), —CH₂ CH₂CH₂CH₂CH₂NH₂, —CH₂CH₂CH(NH₂)CH₃, —CH₂CH(NH₂)CH₂CH₂CH₃, —CH₂CH(NH₂)CH₂CH₃ and —CH₂C(CH₃)₂(CH₂NH₂). Representative examples of a C₁-C₅ alkyl substituted with a —C(O)NH₂ group include, but are not limited to —CH₂C(O)NH₂, —CH₂CH₂C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃ and —C(CH₃)₂CH₂C(O)NH₂. Representative examples of a C₁-C₅ alkyl substituted with an —OH group include, but are not limited to —CH₂OH, —CH₂CH₂OH, —CH₂CH₂CH₂OH, —CH₂CH₂CH₂CH₂OH, —CH₂CH₂CH₂CH₂CH₂OH, —CH₂CH(OH)CH₃, —CH₂CH(OH)CH₂CH₃, —CH(OH)CH₂CH₃ and —C(CH₃)₂CH₂OH. Representative examples of a C₁-C₅ alkyl group substituted with a —C(O)OH group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)₂CH₂COOH.

The term “—(C₂-C₁₀)alkenyl” as used herein, refers to a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

The term “—(C₂-C₁₀) alkynyl” as used herein, refers to a straight chain or branched non-cyclic hydrocarbon having from 2 to 10 carbon atoms and including at lease one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl and the like.

The term “—(C₃-C₈) monocyclic cycloalkyl” as used herein, refers to a saturated cyclic hydrocarbon having from 3 to 8 carbon atoms. Representative (C₃-C₈)cycloalkyls include -cyclopropyl, -cyclobutyl, -cyclopentyl, -cyclohexyl, -cycloheptyl and -cyclooctyl.

The term “—(C₈-C₁₄) bicyclic cycloalkyl” as used herein, refers to a bi-cyclic hydrocarbon ring system having from 8 to 14 carbon atoms and at least one saturated cyclic alkyl ring. Representative —(C₈-C₁₄) bicycloalkyls include -indanyl, -1,2,3,4-tetrahydronaphthyl, -5,6,7,8-tetrahydronaphthyl, -perhydronaphthyl and the like.

The term “—(C₅-C₈) monocyclic cycloalkenyl” as used herein, refers to a cyclic non-aromatic hydrocarbon having at least one carbon-carbon double bond in the cyclic system and from 5 to 8 carbon atoms. Representative (C₅-C₈) monocyclic cycloalkenyls include -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl and the like.

The term “—(C₈-C₁₄) bicyclic cycloalkenyl” as used herein, refers to a bi-cyclic hydrocarbon ring system having at least one carbon-carbon double bond in each ring and from 8 to 14 carbon atoms. Representative —(C₈-C₁₄) bicyclic cycloalkenyls include -indenyl, -pentalenyl, -naphthalenyl, -azulenyl, -heptalenyl, -1,2,7,8-tetrahydronaphthalenyl and the like.

A “3- to 7-membered monocyclic heterocycle” refers to a monocyclic 3- to 7-membered aromatic or non-aromatic monocyclic cycloalkyl in which 1-4 of the ring carbon atoms have been independently replaced with a N, O or S atom. The 3- to 7-membered monocyclic heterocycles can be attached via a nitrogen, sulfur, or carbon atom. Representative examples of a 3- to 7-membered monocyclic heterocycle group include, but are not limited to, piperidinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl.

A “7- to 10-membered bicyclic heterocycle” refers to a bicyclic 7- to 10-membered aromatic or non-aromatic bicyclic cycloalkyl in which 1-4 of the ring carbon atoms have been independently replaced with a N, O or S atom. The 7- to 10-membered bicyclic heterocycles can be attached via a nitrogen, sulfur, or carbon atom. Representative examples of a 7- to 10-membered bicyclic heterocycle group include, but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl.

A “nitrogen-containing 3- to 7-membered monocyclic heterocycle” refers to a 3- to 7-membered monocyclic heterocycle, defined above, which contains at least one ring nitrogen atom. The nitrogen-containing 3- to 7-membered monocyclic heterocycles can be attached via a nitrogen, sulfur, or carbon atom. Representative examples of nitrogen-containing-3- to 7-membered monocyclic heterocycles include, but are not limited to, piperidinyl, piperazinyl, pyrrolyl, oxazinyl, thiazinyl, diazinyl, triazinyl, tetrazinyl, imidazolyl, tetrazolyl, pyrrolidinyl, isoxazolyl, pyridinyl, oxazolyl, thiazolyl, pyrazolyl, triazolyl, pyrimidinyl, and morpholinyl.

A “nitrogen-containing 7- to 10-membered bicyclic heterocycle” refers to a 7- to 10-membered bicyclic heterocycle, defined above, which contains at least one ring nitrogen atom. The nitrogen-containing 7- to 10-membered bicyclic heterocycles can be attached via a nitrogen, sulfur, or carbon atom. Representative nitrogen-containing 7- to 10-membered bicyclic heterocycles include -quinolinyl, -isoquinolinyl, -chromonyl, -indolyl, -isoindolyl, -indolizinyl, -indazolyl, -purinyl, -4H-quinolizinyl, -isoquinolyl, -quinolyl, -phthalazinyl, -naphthyridinyl -carbazolyl, -β-carbolinyl and the like.

The term “glycoside” as used herein refers to a hexose or a pentose sugar forming an α- or β-glycosidic linkage. Representative examples of glycosides include, but are not limited to ribose, deoxyribose, fructose, galactose, glucuronic acid and glucose.

The term “aryl” as used herein, refers to a phenyl or naphthyl group.

The term “animal,” as used herein, includes, but is not limited to, a cow, monkey, horse, sheep, pig, chicken, turkey, quail, cat, dog, mouse, rat, rabbit, guinea pig and human. In one embodiment, an animal is a human.

The phrase “pharmaceutically acceptable salt,” as used herein, is a salt formed from an acid and a basic nitrogen group of one of the Tetracyclic Lactam Derivatives. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, besylate, mesylate, camphor sulfonate, and pamoate (i.e., 1,1′-methylene-bis-(2-OH-3-naphthoate)) salts. The term “pharmaceutically acceptable salt” also refers to a salt prepared from a Tetracyclic Lactam Derivative having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-OHethyl)amine, 2-OH tert-butylamine, or tris-(hydroxymethyl)methylamine, N,N-di-lower alkyl-N-(hydroxy lower alkyl)-amines, such as N,N-dimethyl-N-(2-hydroxyethyl)amine or tri-(2-OHethyl)amine; N-methyl-D-glucamine; and amino acids such as arginine, lysine, and the like. A hydrate is another example of a pharmaceutically acceptable salt.

When a first group is “substituted with one or more” second groups, each of one or more of the first group's hydrogen atoms is replaced with a second group. In one embodiment each carbon atom of a first group is independently substituted with one or two second groups. In another embodiment each carbon atom of a first group is independently substituted with only one second group.

The term “effective amount” when used in connection with a Tetracyclic Lactam Derivative is an amount that is effective to: (a) treat or prevent a Condition; or (b) inhibiting PARP in an in vivo or an in vitro cell.

An “effective amount” when used in connection with another anticancer agent is an amount that is effective for treating or preventing cancer alone or in combination with a Tetracyclic Lactam Derivative. “In combination with” includes administration within the same composition and within separate compositions. In the latter instance, the anticancer agent is administered during a time when the Tetracyclic Lactam Derivative exerts its prophylactic or therapeutic effect, or vice versa.

The term “isolated and purified” as used herein means separated from other components of a reaction mixture or natural source.

The following abbreviations are used herein and have the following meanings: DIEA is diisopropylethylamine, DMF is dimethyl formamide, DMSO is dimethyl sulfoxide, DPPA is diphenylphosphorylazide, Et₃N is triethylamine, EtOH is ethanol, MeOH is methanol, NaH is sodium hydride, NBS is N-bromosuccinimide, PPA is polyphosphoric acid, pyr is pyridine, THF is tetrahydrofuran, and TMZ is temozolomide.

### 4.9 Methods for Making the Tetracyclic Lactam Derivatives

The Tetracyclic Lactam Derivatives can be made using conventional organic synthesis or by the following illustrative methods shown in Schemes 1-4 below.

Scheme 1 below illustrates a method useful for making the Tetracyclic Lactam Derivatives of Formula (I), wherein R¹-R¹¹ are as defined above for the compounds of Formula (I).

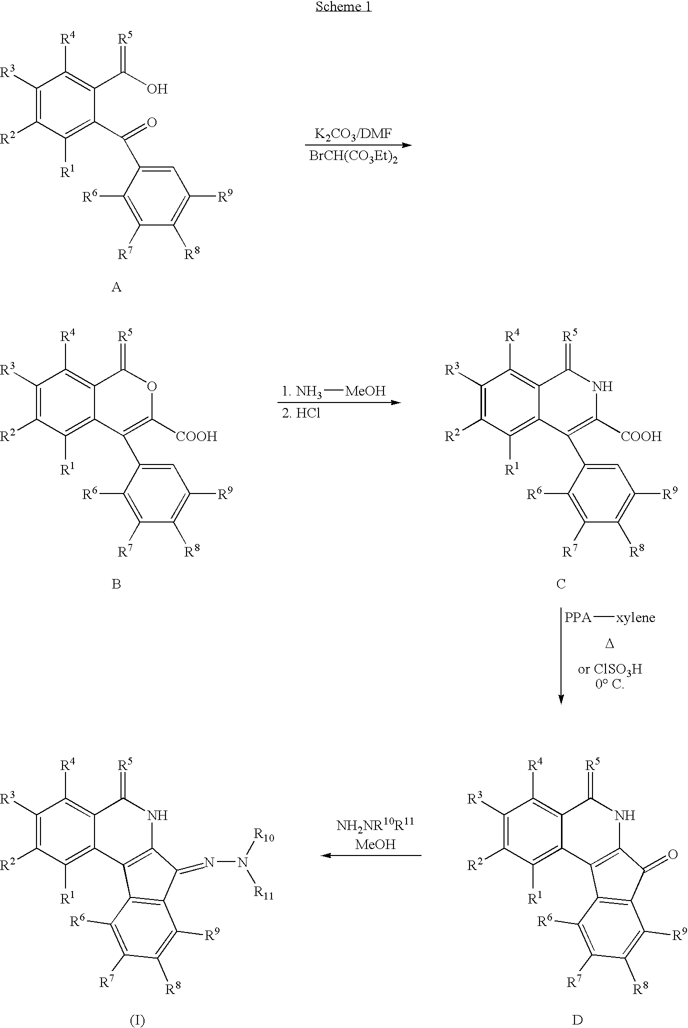

A benzophenone of formula A can be cyclized to the bicyclic intermediates of formula B using bromo ethyl malonate in the presence of potassium carbonate. The intermediates of formula B can then be converted to the lactam intermediates of formula C in the presence of ammonia in methanol. Fridel-Crafts mediated ring closure of C provides the tetracyclic ketone intermediates of formula D which can be coupled with a hydrazine to provide the Tetracyclic Lactam Derivatives of Formula (I).

Scheme 2 further illustrates the formation of particular —NR₁₀R₁₁ groups of Formula (I). Reacting the tetracyclic ketone intermediate of formula D with the particular hydrazines set forth in Scheme 2 in the presence of a suitable acid, such as acetic acid or hydrochloric acid, results in the formation of compounds 1, 7, 105, and 106, respectively.

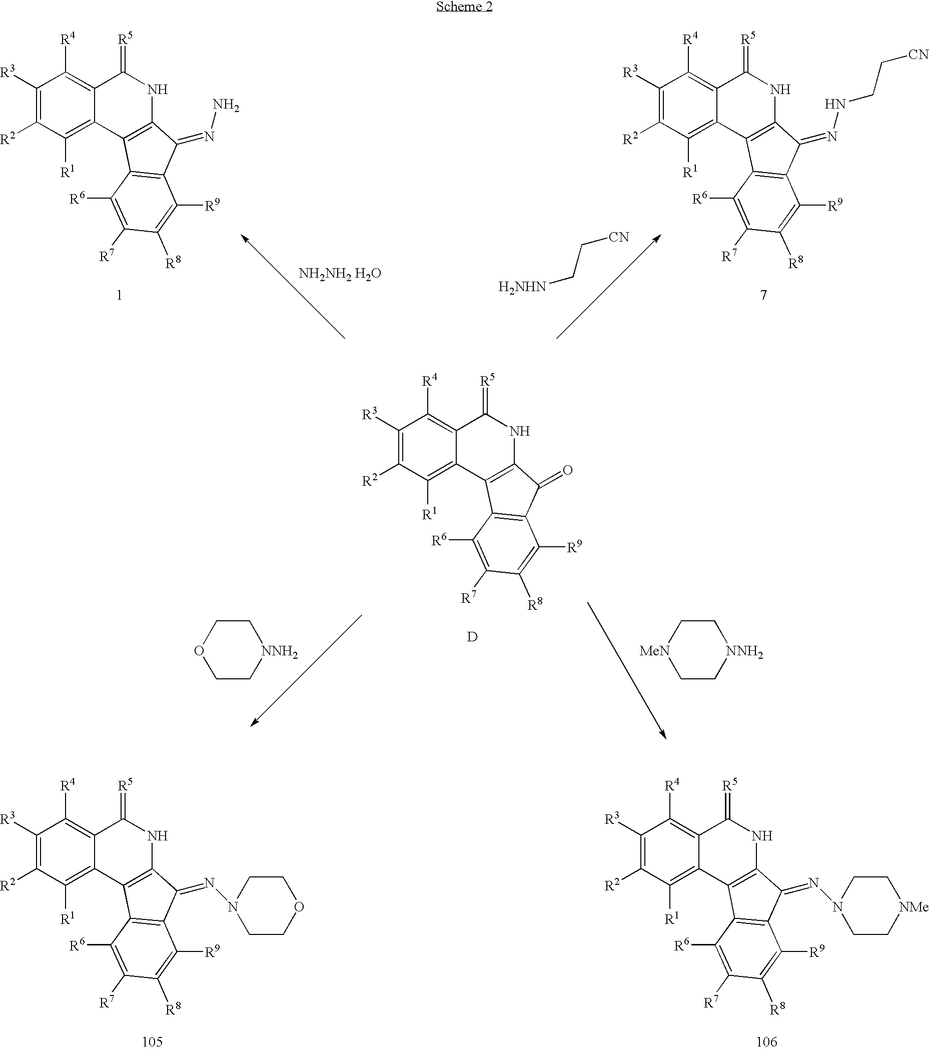

Scheme 3 below illustrates a method useful for making the Tetracyclic Lactam Derivatives of Formula (II), wherein R¹-R¹⁰ are as defined above for the compounds of Formula

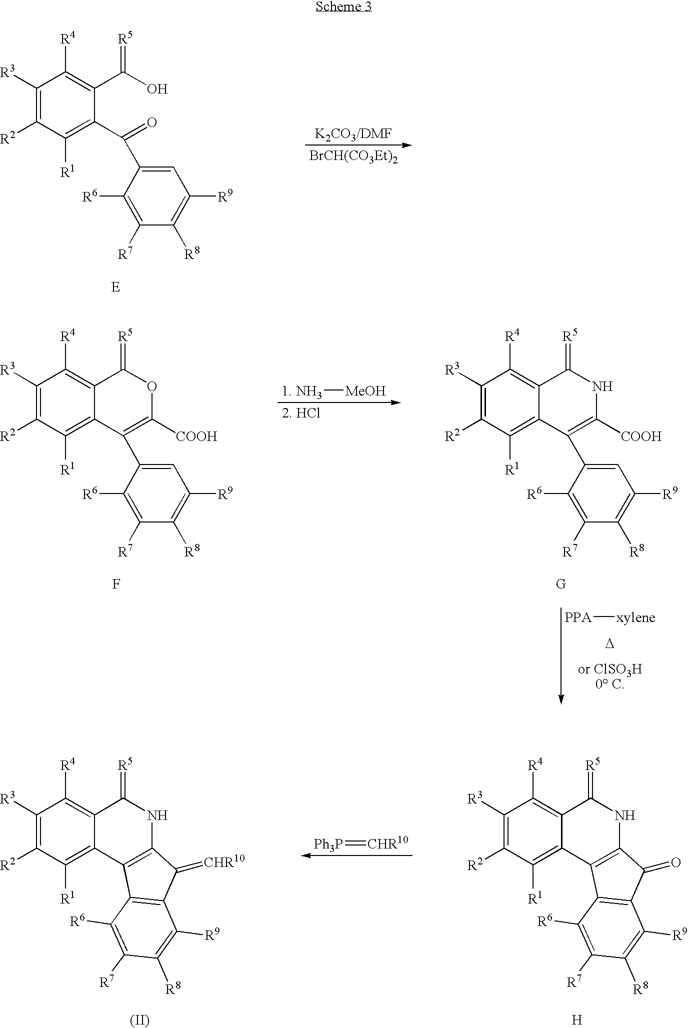

A ketone of formula E can be cyclized to the bicyclic intermediate of formula F using bromo ethyl malonate in the presence of potassium carbonate. The intermediates of formula F can then be converted to the lactam intermediates of formula G in the presence of ammonia in methanol. Fridel-Crafts mediated ring closure of G provides the tetracyclic ketone intermediates of formula H, which can be reacted with a phosphonate or phosphorus ylide via a Wittig procedure (see March, J, Advanced Organic Chemistry, Reactions, Mechanisms, and Structure, p. 956-963 (4^{th} Ed. 1992)) to provide the Tetracyclic Lactam Derivatives of Formula (II). Alternatively, the tetracyclic ketone intermediates of formula H can be reacted with a reagent such as R¹⁰CH₂Li followed by dehydration to provide the Tetracyclic Lactam Derivatives of Formula (II).

The Tetracyclic Lactam Derivatives of Formula (III) can be made using the methods described below in Scheme 4, wherein R¹-R¹⁰ are as defined above for the compounds of Formula (III).

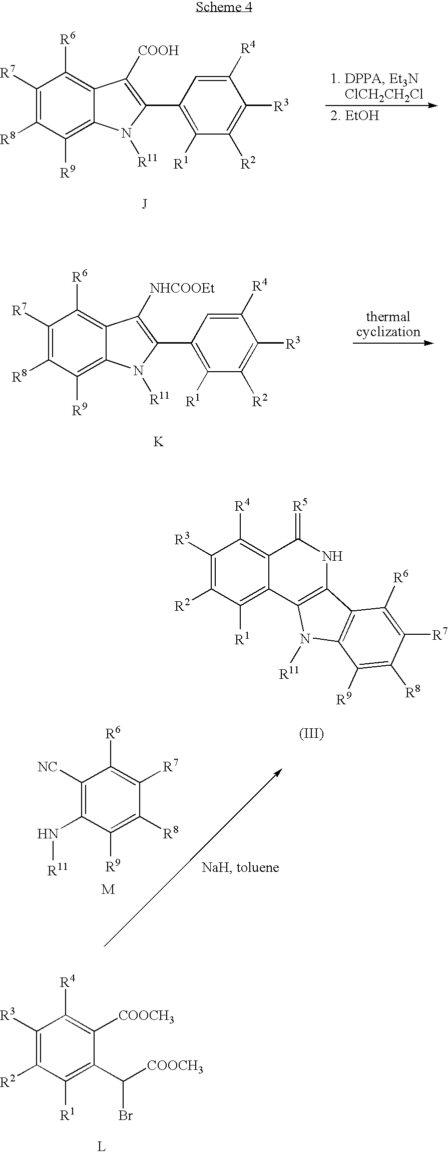

The carboxylic acid group of a compound of formula J (see Wacker et al., Tet. Lett., 43:5189-5191, 2002; and Bourdais et al, J. Het. Chem., 12:1111-1115, 1975, for methods useful to make compounds of formula J) can be coupled with DPPA to provide the corresponding carbonate intermediates of formula K, which can then be thermally cyclized by refluxing the compounds of formula K in diphenyl ether or by heating the neat compounds of formula K to between 300° C. and 350° C. to provide the Tetracyclic Lactam Derivatives of Formula (III).

Alternatively, the Tetracyclic Lactam Derivatives of Formula (III) can be made using a one pot coupling/cyclization process by reacting a bromo intermediate of formula L with an aromatic nitrile of formula M in the presence of sodium hydride.

A Tetracyclic Lactam Derivative of Formula (IV), (V), or (VI) can be made by reacting a Tetracyclic Lactam Derivative of Formula (I), (II), or (III) respectively, with a compound having the formula: (a) R¹³X, where X is a leaving group such as halogen; or (b) R¹³—C(O)—O—C(O)—R¹³, under conditions well-known to those skilled in the art of organic synthesis.

### 4.10 Therapeutic Uses of the Tetracyclic Lactam Derivatives

The invention also includes pharmaceutical compositions comprising an effective amount of a Tetracyclic Lactam Derivative and a physiologically acceptable carrier or vehicle.

In accordance with the invention, the Tetracyclic Lactam Derivatives are administered to an animal in need of treatment or prevention of a Condition.

### 4.10.1 Treatment or Prevention of an Inflammatory Disease

The Tetracyclic Lactam Derivatives can be used to treat or prevent an inflammatory disease. Inflammatory diseases can arise where there is an inflammation of the body tissue. These include local inflammatory responses and systemic inflammation. Examples of inflammatory diseases treatable or preventable using the Tetracyclic Lactam Derivatives include, but are not limited to, organ transplant rejection; chronic inflammatory diseases of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung diseases such as asthma, adult respiratory distress syndrome, and chronic obstructive airway disease; inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; tuberculosis; leprosy; inflammatory diseases of the kidney including uremic complications, glomerulonephritis and nephrosis; inflammatory diseases of the skin including dermatitis, sclerodermatitis, psoriasis and eczema; inflammatory diseases of the central nervous system, including chronic demyelinating diseases of the nervous system, multiple sclerosis, AIDS-related neurodegeneration and Alzheimers disease, infectious meningitis, encephalomyelitis, Parkinson's disease, Huntington's disease, amyotrophic lateral sclerosis and viral or autoimmune encephalitis; immune-complex vasculitis; systemic lupus erythematosus (SLE); inflammatory diseases of the heart such as cardiomyopathy, ischemic heart disease, hypercholesterolemia, and atherosclerosis; as well as various other diseases that can have significant inflammatory components, including preeclampsia, chronic liver failure, and brain and spinal cord trauma. The inflammatory disease can also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

### 4.10.2 Treatment or Prevention of a Reperfusion Injury

The Tetracyclic Lactam Derivatives can be used to treat or prevent a reperfusion injury. Reperfusion refers to the process whereby blood flow in the blood vessels is resumed following ischemia, such as occurs following constriction or obstruction of the vessel. Reperfusion injury can result following a naturally occurring episode, such as a myocardial infarction, stroke, or during a surgical procedure where blood flow in vessels is intentionally or unintentionally blocked. Examples of reperfusion injuries treatable or preventable using the Tetracyclic Lactam Derivatives include, but are not limited to, intestinal reperfusion injury, myocardial reperfusion injury, and reperfusion injury resulting from cardiopulmonary bypass surgery, aortic aneurysm repair surgery, carotid endarterectomy surgery, or hemorrhagic shock.

In one embodiment, the reperfusion injury results from cardiopulmonary bypass surgery, aortic aneurysm repair surgery, carotid endarterectomy surgery, or hemorrhagic shock.

In one embodiment, the reperfusion injury is a reoxygenation injury resulting from surgery, particularly that relating to organ transplantation.

### 4.10.3 Treatment or Prevention of a Reoxygenation Injury Resulting from Organ Transplantation

The Tetracyclic Lactam Derivatives can be used to treat or prevent a reoxygenation injury resulting from surgery, particularly that relating to organ transplantation. Examples of reoxygenation injuries treatable or preventable using the Tetracyclic Lactam Derivatives include, but are not limited to, transplantation of the following organs: heart, lung, liver, kidney, pancreas, intestine, and cornea.

In one embodiment, a reoxygenation injury resulting from organ transplantation occurs during the organ transplantation.

### 4.10.4 Treatment or Prevention of an Ischemic Condition

The Tetracyclic Lactam Derivatives can be used to treat or prevent an ischemic condition. Examples of ischemic conditions treatable or preventable using the Tetracyclic Lactam Derivatives include, but are not limited to, stable angina, unstable angina, myocardial ischemia, hepatic ischemia, mesenteric artery ischemia, intestinal ischemia, critical limb ischemia, chronic critical limb ischemia, cerebral ischemia, acute cardiac ischemia, and an ischemic disease of the central nervous system, such as stroke or cerebral ischemia.

In one embodiment, the ischemic condition is myocardial ischemia, stable angina, unstable angina, stroke, ischemic heart disease or cerebral ischemia.

### 4.10.5 Treatment or Prevention of Renal Failure

The Tetracyclic Lactam Derivatives can be used to treat or prevent renal failure. In one embodiment the renal failure is chronic renal failure. In another embodiment, the renal failure is acute renal failure.

### 4.10.6 Treatment or Prevention of a Vascular Disease

The Tetracyclic Lactam Derivatives can be used to treat or prevent a vascular disease. Examples of vascular diseases treatable or preventable using the Tetracyclic Lactam Derivatives include, but are not limited to, peripheral arterial occlusion, thromboangitis obliterans, Reynaud's disease and phenomenon, acrocyanosis, erythromelalgia, venous thrombosis, varicose veins, arteriovenous fistula, lymphedema, and lipedema.

### 4.10.7 Treatment or Prevention of a Cardiovascular Disease

The Tetracyclic Lactam Derivatives can be used to treat or prevent a cardiovascular disease. Examples of cardiovascular diseases treatable or preventable using the Tetracyclic Lactam Derivatives include chronic heart failure, atherosclerosis, congestive heart failure, circulatory shock, cardiomyopathy, cardiac transplant, myocardial infarction, and a cardiac arrhythmia, such as atrial fibrillation, supraventricular tachycardia, atrial flutter, and paroxysmal atrial tachycardia.

In one embodiment, the cardiovascular disease is chronic heart failure.

In another embodiment, the cardiovascular disease is a cardiac arrhythmia.

In still another embodiment, the cardiac arrhythmia is atrial fibrillation, supraventricular tachycardia, atrial flutter or paroxysmal atrial tachycardia.

### 4.10.8 Treatment or Prevention of Diabetes or Diabetic Complication

The Tetracyclic Lactam Derivatives can be used to treat or prevent diabetes mellitus or its complications. Examples of diabetes treatable or preventable or preventable using the Tetracyclic Lactam Derivatives include, but are not limited to, Type I diabetes (Insulin Dependent Diabetes Mellitus), Type II diabetes (Non-Insulin Dependent Diabetes Mellitus), gestational diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), Type A insulin resistance syndrome, Type B insulin resistance syndrome, lipatrophic diabetes, and diabetes induced by β-cell toxins.

The Tetracyclic Lactam Derivatives can be used to treat or prevent a diabetic complication. Examples of diabetic mellitus or its complications that are treatable or preventable or preventable using the Tetracyclic Lactam Derivatives include, but are not limited to, diabetic cataract, glaucoma, retinopathy, nephropathy, (such as microaluminuria and progressive diabetic nephropathy), polyneuropathy, gangrene of the feet, atherosclerotic coronary arterial disease, peripheral arterial disease, nonketotic hyperglycemic-hyperosmolar coma, mononeuropathy, autonomic neuropathy, a skin or mucous membrane complication (such as an infection, a shin spot, a candidal infection or necrobiosis lipoidica diabeticorumobesity), hyperlipidemia, hypertension, syndrome of insulin resistance, coronary artery disease, retinopathy, diabetic neuropathy, polyneuropathy, mononeuropathy, autonomic neuropathy, a foot ulcer, a joint disease, a fungal infection, and a bacterial infection, and cardiomyopathy.

### 4.10.9 Treatment or Prevention of Parkinson's Disease

The Tetracyclic Lactam Derivatives can be used to treat or prevent Parkinson's disease.

### 4.10.10 Treatment or Prevention of Cancer

The Tetracyclic Lactam Derivatives can be used to treat or prevent cancer. Examples of cancers treatable or preventable using the Tetracyclic Lactam Derivatives include, but are not limited to, the cancers disclosed below in Table 1 and metastases thereof.

TABLE 1

Solid tumors, including but not limited to:
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
skin cancer
melanoma
neuroblastoma
retinoblastoma
blood-borne cancers, including but not limited to:
acute lymphoblastic leukemia (“ALL”)
acute lymphoblastic B-cell leukemia
acute lymphoblastic T-cell leukemia
acute myeloblastic leukemia (“AML”)
acute promyelocytic leukemia (“APL”)
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia (“CML”)
chronic lymphocytic leukemia (“CLL”)
hairy cell leukemia
multiple myeloma
acute and chronic leukemias:
lymphoblastic
myelogenous
lymphocytic
myelocytic leukemias
Lymphomas:
Hodgkin's disease
non-Hodgkin's Lymphoma
Multiple myeloma
Waldenstrom's macroglobulinemia
Heavy chain disease
Polycythemia vera
CNS and brain cancers:
glioma
pilocytic astrocytoma
astrocytoma
anaplastic astrocytoma
glioblastoma multiforme
medulloblastoma
craniopharyngioma
ependymoma
pinealoma
hemangioblastoma
acoustic neuroma
oligodendroglioma
meningioma
vestibular schwannoma
adenoma
metastatic brain tumor
meningioma
spinal tumor
medulloblastoma

In one embodiment the cancer is lung cancer, breast cancer, colorectal cancer, prostate cancer, a leukemia, a lymphoma, a non-Hodgkin's lymphoma, a skin cancer, a brain cancer, a cancer of the central nervous system, ovarian cancer, uterine cancer, stomach cancer, pancreatic cancer, esophageal cancer, kidney cancer, liver cancer, or a head and neck cancer.

In another embodiment the cancer is metastatic cancer.

In still another embodiment, the animal in need of treatment has previously undergone or is presently undergoing treatment for cancer. Such previous treatments include, but are not limited to, prior chemotherapy, radiation therapy, surgery or immunotherapy, such as cancer vaccines.

The Tetracyclic Lactam Derivatives are also useful for the treatment or prevention of a cancer caused by a virus. Such viruses include human papilloma virus, which can lead to cervical cancer (see, e.g., Hemandez-Avila et al., Archives of Medical Research (1997) 28:265-271); Epstein-Barr virus (EBV), which can lead to lymphoma (see, e.g., Herrmann et al., J Pathol (2003) 199(2): 140-5); hepatitis B or C virus, which can lead to liver carcinoma (see, e.g., El-Serag, J Clin Gastroenterol (2002) 35(5 Suppl 2):S72-8); human T cell leukemia virus (HTLV)-I, which can lead to T-cell leukemia (see e.g., Mortreux et al., Leukemia (2003) 17(1):26-38); human herpesvirus-8 infection, which can lead to Kaposi's sarcoma (see, e.g., Kadow et al., Curr Opin Investig Drugs (2002) 3(11): 1574-9); and Human Immune deficiency Virus (HIV) infection, which can lead to cancer as a consequence of immunodeficiency (see, e.g., Dal Maso et al., Lancet Oncol (2003) 4(2): 110-9).

The Tetracyclic Lactam Derivatives of the Invention can also be administered to prevent the progression of a cancer, including but not limited to the cancers listed in Table 1. Such prophylactic use includes that in which non-neoplastic cell growth consisting of hyperplasia, metaplasia, or most particularly, dysplasia has occurred.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from an animal, can indicate the desirability of prophylactic/therapeutic administration of a Tetracyclic Lactam Derivative. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are treatable or preventable according to the present methods.

In another embodiment, fibrocystic disease (cystic hyperplasia, mammary dysplasia, particularly adenosis (benign epithelial hyperplasia)) are treatable or preventable according to the present methods.

In other embodiments, an animal that exhibits one or more of the following predisposing factors for malignancy can be administered an amount of a Tetracyclic Lactam Derivative which is effective to treat or prevent cancer: a chromosomal translocation associated with a malignancy (e.g., the Philadelphia chromosome for chronic myelogenous leukemia, t(14;18) for follicular lymphoma); familial polyposis or Gardner's syndrome; benign monoclonal gammopathy; a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome, and exposure to carcinogens (e.g., smoking, second-hand smoke exposure, and inhalation of or contacting with certain chemicals).

In another specific embodiment, a Tetracyclic Lactam Derivatives is administered to a human patient to prevent progression to breast, colon, ovarian, or cervical cancer.

In one embodiment, the present methods for treating cancer or preventing cancer further comprise administering another anticancer agent.

In one embodiment, the present invention provides methods for treating or preventing cancer in a animal, the method comprising the administration of an effective amount of: (i) a Tetracyclic Lactam Derivative, and (ii) another anticancer agent.

In one embodiment, a Tetracyclic Lactam Derivative and another anticancer agent are administered in doses commonly employed when such agents are used as monotherapy for the treatment of cancer.

In another embodiment, a Tetracyclic Lactam Derivative and another anticancer agent act synergistically and are administered in doses that are less than the doses commonly employed when such agents are used as monotherapy for the treatment of cancer.

The dosage of a Tetracyclic Lactam Derivative and another anticancer agent administered as well as the dosing schedule can depend on various parameters, including, but not limited to, the cancer being treated, the patient's general health, and the administering physician's discretion.

A Tetracyclic Lactam Derivative can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other anticancer agent to a animal in need thereof. In various embodiments, a Tetracyclic Lactam Derivative and another anticancer agent are administered 1 minute apart, 10 minutes apart, 30 minutes apart, less than 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, or no more than 48 hours apart.

In one embodiment, a Tetracyclic Lactam Derivative and another anticancer agent are administered with 3 hours. In another embodiment, a Tetracyclic Lactam Derivative and another anticancer agent are administered 1 minute to 24 hours apart.

In one embodiment, an effective amount of a Tetracyclic Lactam Derivative and an effective amount of another anticancer agent are present in the same composition. In one embodiment, this composition is useful for oral administration. In another embodiment, this composition is useful for intravenous administration.

Cancers that can be treated or prevented by administering a Tetracyclic Lactam Derivative and another anticancer agent include, but are not limited to, the list of cancers set forth in Table 1.

In one embodiment, the cancer is brain cancer.

In specific embodiments, the brain cancer is pilocytic astrocytoma, astrocytoma, anaplastic astrocytoma, glioblastoma multiforme or a metastatic brain cancer.

In a specific embodiment, the cancer is melanoma.

In one embodiment, the cancer is metastatic melanoma.

### 4.10.11 Therapeutic/Prophylactic Administration and Compositions of the Invention

Due to their activity, the Tetracyclic Lactam Derivatives are advantageously useful in veterinary and human medicine. As described above, the Tetracyclic Lactam Derivatives are useful for treating or preventing a Condition in an animal in need thereof.

When administered to an animal, the Tetracyclic Lactam Derivatives can be administered as a component of a composition that comprises a physiologically acceptable carrier or vehicle. The present compositions, which comprise a Tetracyclic Lactam Derivative, can be administered orally. The Tetracyclic Lactam Derivatives of the invention can also be administered by any other convenient route, for example, by infusion or bolus injection, by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the Tetracyclic Lactam Derivatives into the bloodstream. The mode of administration can be left to the discretion of the practitioner.

In one embodiment, the Tetracyclic Lactam Derivatives are administered orally.

In other embodiments, it can be desirable to administer the Tetracyclic Lactam Derivatives locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Tetracyclic Lactam Derivatives into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon oar, synthetic pulmonary surfactant. In certain embodiments, the Tetracyclic Lactam Derivatives can be formulated as a suppository, with traditional binders and excipients such as triglycerides.

In another embodiment the Tetracyclic Lactam Derivatives can be delivered in a vesicle, in particular a liposome (see Langer, Science 249:1527-1533 (1990) and Treat or prevent et al., Liposomes in the Therapy of Infectious Disease and Cancer 317-327 and 353-365 (1989)).

In yet another embodiment the Tetracyclic Lactam Derivatives can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the Tetracyclic Lactam Derivatives, e.g., the spinal column, brain, skin, lung, or gastrointestinal tract, thus requiring only a fraction of the systemic dose.

The present compositions can optionally comprise a suitable amount of a pharmaceutically acceptable excipient so as to provide the form for proper administration to the animal.

Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia; gelatin, starch paste, talc, keratin, colloidal silica, urea and the like. In addition, auxiliary, stabilizing, thickening, lubricating, and coloring agents can be used. In one embodiment the pharmaceutically acceptable excipients are sterile when administered to an animal. Water is a particularly useful excipient when the Tetracyclic Lactam Derivative is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills; pellets, capsules, capsules containing liquids, powders, sustained-release formulations, suppositories, emulsions, aerosols, sprays, suspensions, or any other form suitable for use. In one embodiment the composition is in the form of a capsule (see e.g. U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In one embodiment the Tetracyclic Lactam Derivatives are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving a Tetracyclic Lactam Derivative are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the Tetracyclic Lactam Derivatives can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaien to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the Tetracyclic Lactam Derivatives are to be administered by infusion, they can be dispensed, for example, with an infusion bottle containing sterile pharmaceutical grade water or saline. Where the Tetracyclic Lactam Derivatives are administered by injection, an ampule of sterile water for injection or saline can be provided so that the ingredients can be mixed prior to administration.

The Tetracyclic Lactam Derivatives can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of a Tetracyclic Lactam Derivative to treat or prevent the Condition in a minimal amount of time. Advantages of controlled- or sustained-release compositions include extended activity of the drug, reduced dosage frequency, and increased patient compliance. In addition, controlled- or sustained-release compositions can favorably affect the time of onset of action or other characteristics, such as blood levels of the Tetracyclic Lactam Derivative, and can thus reduce the occurrence of adverse side effects.

Controlled- or sustained-release compositions can initially release an amount of a Tetracyclic Lactam Derivative that promptly produces the desired therapeutic or prophylactic effect, and gradually and continually release other amounts of the Tetracyclic Lactam Derivative to maintain this level of therapeutic or prophylactic effect over an extended period of time. To maintain a constant level of the Tetracyclic Lactam Derivative in the body, the Tetracyclic Lactam Derivative can be released from the dosage form at a rate that will replace the amount of Tetracyclic Lactam Derivative being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of the Tetracyclic Lactam Derivative that is effective in the treatment or prevention of a Condition can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each patient's circumstances in view of, e.g., published clinical studies. Suitable effective dosage amounts, however, range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one Tetracyclic Lactam Derivative is administered, the effective dosage amounts correspond to the total amount administered.

Compositions can be prepared according to conventional mixing, granulating or coating methods, respectively, and the present compositions can contain from about 0.1% to about 99%, in one embodiment, from about 1% to about 70% of the Tetracyclic Lactam Derivative by weight or volume.

The dosage regimen utilizing the Tetracyclic Lactam Derivative can be selected in accordance with a variety of factors including type, species, age, weight, sex, and medical condition of the animal; the severity of the condition to be treated; the route of administration; the renal or hepatic function of the animal; and the particular Tetracyclic Lactam Derivative employed. A person skilled in the art can readily determine and prescribe the effective amount of the Tetracyclic Lactam Derivative useful for treating or preventing a Condition.

The Tetracyclic Lactam Derivative can be administered in a single daily dose, or the total daily dosage can be administered in divided doses of two, three or four times daily. Furthermore, a Tetracyclic Lactam Derivative can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of a Tetracyclic Derivative ranges from about 0.1% to about 15%, w/w or w/v.

In one embodiment, the compositions comprise an amount of each of a Tetracyclic Lactam Derivative and another anticancer agent which together are effective to treat or prevent cancer. In another embodiment, the amount of Tetracyclic Lactam Derivative and another anticancer agent is at least about 0.01% of the combined combination chemotherapy agents by weight of the composition. When intended for oral administration, this amount can be varied from about 0.1% to about 80% by weight of the composition. Some oral compositions can comprise from about 4% to about 50% of a Tetracyclic Lactam Derivative and another anticancer agent. Other compositions of the present invention are prepared so that a parenteral dosage unit contains from about 0.01% to about 2% by weight of the composition.

The Tetracyclic Lactam Derivatives can be assayed in vitro or in vivo for the desired therapeutic or prophylactic activity prior to use in humans. Animal model systems can be used to demonstrate safety and efficacy.

The present methods for treating or preventing a Condition in an animal in need thereof can further comprise administering another prophylactic or therapeutic agent to the animal being administered a Tetracyclic Lactam Derivative. In one embodiment the other prophylactic or therapeutic agent is administered in an effective amount. The other prophylactic or therapeutic agent includes, but is not limited to, an anti-inflammatory agent, an anti-renal failure agent, an anti-diabetic agent, an anti-cardiovascular disease agent, an antiemetic agent, a hematopoietic colony stimulating factor, an anxiolytic agent, an analgesic agent, and an anti-cancer agent.

In one embodiment, the Tetracyclic Lactam Derivative can be administered prior to, concurrently with, or after an anti-inflammatory agent, or on the same day, or within 1 hour, 2 hours, 12 hours, 24 hours, 48 hours or 72 hours of each other.

In another embodiment, the Tetracyclic Lactam Derivative can be administered prior to, concurrently with, or after an anti-renal failure agent, or on the same day, or within 1 hour, 2 hours, 12 hours, 24 hours, 48 hours or 72 hours of each other.

In still another embodiment, the Tetracyclic Lactam Derivative can be administered prior to, concurrently with, or after an anti-diabetic agent, or on the same day, or within 1 hour, 2 hours, 12 hours, 24 hours, 48 hours or 72 hours of each other.

In yet another embodiment, the Tetracyclic Lactam Derivative can be administered prior to, concurrently with, or after an anti-cardiovascular disease agent, or on the same day, or within 1 hour, 2 hours, 12 hours, 24 hours, 48 hours or 72 hours of each other.

In a further embodiment, the Tetracyclic Lactam Derivative can be administered prior to, concurrently with, or after an antiemetic agent, or on the same day, or within 1 hour, 2 hours, 12 hours, 24 hours, 48 hours or 72 hours of each other.

In another embodiment, the Tetracyclic Lactam Derivative can be administered prior to, concurrently with, or after a hematopoietic colony stimulating factor, or on the same day, or within 1 hour, 2 hours, 12 hours, 24 hours, 48 hours, 72 hours, 1 week, 2 weeks, 3 weeks or 4 weeks of each other.

In still embodiment, the Tetracyclic Lactam Derivative can be administered prior to, concurrently with, or after an opioid or non-opioid analgesic agent, or on the same day, or within 1 hour, 2 hours, 12 hours, 24 hours, 48 hours or 72 hours of each other.

In yet another embodiment, the Tetracyclic Lactam Derivative can be administered prior to, concurrently with, or after an anxiolytic agent, or on the same day, or within 1 hour, 2 hours, 12 hours, 24 hours, 48 hours or 72 hours of each other.

Effective amounts of the other therapeutic agents are known to those skilled in the art. However, it is well within the skilled artisan's purview to determine the other therapeutic agent's optimal effective amount range. In one embodiment of the invention, where, another therapeutic agent is administered to an animal, the effective amount of the Tetracyclic Lactam Derivative is less than its effective amount would be where the other therapeutic agent is not administered. In this case, without being bound by theory, it is believed that the Tetracyclic Lactam Derivatives and the other therapeutic agent act synergistically to treat or prevent a Condition.

In one embodiment the other anti-inflammatory agents include but are not limited to adrenocorticosteroids, such as cortisol, cortisone, fludrocortisone, prednisone, prednisolone, 6a-methylprednisolone, triamcinolone, betamethasone, and dexamethasone; and non-steroidal anti-inflammatory agents (NSAIDs), such as aspirin, acetaminophen, indomethacin, sulindac, tolmetin, diclofenac, ketorolac, ibuprofen, naproxen, flurbiprofen, ketoprofen, fenoprofen, oxaprozin, mefenamic acid, meclofenamic acid, piroxicam, meloxicam, nabumetone, rofecoxib, celecoxib, etodolac, and nimesulide.

In one embodiment the other anti-renal failure agents include but are not limited to ACE (angiotensin-converting enzyme) inhibitors, such as captopril, enalaprilat, lisinopril, benazepril, fosinopril, trandolapril, quinapril, and ramipril; diuretics, such as mannitol, glycerin, furosemide, toresemide, tripamide, chlorothiazide, methyclothiazide, indapamide, amiloride, and spironolactone; and fibric acid agents, such as clofibrate, gemfibrozil, fenofibrate, ciprofibrate, and bezafibrate.

In one embodiment the other anti-diabetic agents include but are not limited to glucagons; somatostatin; diazoxide; sulfonylureas, such as tolbutamide, acetohexamide, tolazamide, chloropropamide, glybenclamide, glipizide, gliclazide, and glimepiride; insulin secretagogues, such as repaglinide, and nateglinide; biguanides, such as metformin and phenformin; thiazolidinediones, such as pioglitazone, rosiglitazone, and troglitazone; and α-glucosidase inhibitors, such as acarbose and miglitol.

In one embodiment the other anti-cardiovascular disease agents include but are not limited to carnitine; thiamine; and muscarinic receptor antagonists, such as atropine, scopolamine, homatropine, tropicamide, pirenzipine, ipratropium, tiotropium, and tolterodine.

The other therapeutic agent can also be an agent useful for reducing any potential side effect of a Tetracyclic Lactam Derivatives. For example, the other therapeutic agent can be an antiemetic agent. Examples of useful antiemetic agents include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, and mixtures thereof.

The Tetracyclic Lactam Derivative and the other anticancer agent can act additively or synergistically. A synergistic use of a Tetracyclic Lactam Derivative and another anticancer agent might allow the use of lower dosages of one or more of these agents and/or less frequent administration of said agents to an animal with cancer. The ability to utilize lower dosages of a Tetracyclic Lactam Derivative and/or additional anticancer agents and/or to administer said agents less frequently can reduce the toxicity associated with the administration of said agents to an animal without reducing the efficacy of said agents in the treatment of cancer. In addition, a synergistic effect might result in the improved efficacy of these agents in the treatment of cancer and/or the reduction of adverse or unwanted side effects associated with the use of either agent alone.

In one embodiment, the Tetracyclic Lactam Derivative and the anticancer agent can act synergistically when administered in doses typically employed when such agents are used as monotherapy for the treatment of cancer. In another embodiment, the Tetracyclic Lactam Derivative and the anticancer agent can act synergistically when administered in doses that are less than doses typically employed when such agents are used as monotherapy for the treatment of cancer.

In one embodiment, the administration of an effective amount of a Tetracyclic Lactam Derivative and an effective amount of another anticancer agent inhibits the resistance of a cancer to the other anticancer agent. In one embodiment, the cancer is a tumor.

Suitable additional anticancer agents useful in the methods and compositions of the present invention include, but are not limited to, temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one embodiment, the other anticancer agent is, but is not limited to, a drug listed in Table 2.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| | Procarbazine |
| | Temozolomide |
| Platinum containing complexes: | Cisplatin |
| | Carboplatin |
| | Aroplatin |
| | Oxaliplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxel |
| DNA Topoisomerase Inhibitors | |
| Epipodophyllins: | Etoposide |
| | Teniposide |
| | Topotecan |
| | 9-aminocamptothecin |
| | Camptothecin |
| | Crisnatol |
| Mitomycins: | Mitomycin C |
| | Anti-metabolites |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Fluoxuridine |
| | Doxifluridine |
| | Ralitrexed |
| Cytosine analogs: | Cytarabine (ara C) |
| | Cytosine arabinoside |
| | Fludarabine |
| | Gemcitabine |
| | Capecitabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goscrclin |
| | Leuprolide acetate |
| Anti-androgens: | Flutamide |
| | Bicalutamide |
| Retinoids/Deltoids | Cis-retinoic acid |
| Vitamin A derivative: | All-trans retinoic acid (ATRA-IV) |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| | Interleukin-2 |
| Angiogenesis Inhibitors: | Angiostatin (plasminogen |
| | fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII |
| | fragment) |
| | Fibronectin fragment |
| | Gro-beta |
| | Halofuginone |
| | Heparinases |
| | Heparin hexasaccharide fragment |
| | HMV833 |
| | Human chorionic gonadotropin |
| | (hCG) |
| | IM-862 |
| | Interferon alpha/beta/gamma |
| | Interferon inducible protein (IP- |
| | 10) |
| | Interleukin-12 |
| | Kringle 5 (plasminogen fragment) |
| | Marimastat |
| | Metalloproteinase inhibitors |
| | (TIMPs) |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta |
| | (TGF-β) |
| | Vasculostatin |
| | Vasostatin (calreticulin fragment) |
| | ZD6126 |
| | ZD 6474 |
| | farnesyl transferase inhibitors |
| | (FTI) |
| | Bisphosphonates |
| Antimitotic agents: | Allocolchicine |
| | Halichondrin B |
| | Colchicine |
| | colchicine derivative |
| | dolstatin 10 |
| | Maytansine |
| | Rhizoxin |
| | Thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ ATPase inhibitors: | Thapsigargin |

Other anticancer agents that can be used in the compositions and methods of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine hydrochloride; hydroxyurea; idarubicin hydrochloride; ifosfamide; ilmofosine; interleukin-2 (including recombinant interleukin-2, or rIL2), interferon alfa-2α; interferon alfa-2β; interferon alfa-n1; interferon alfa-n3; interferon beta-Iα; interferon gamma-Iβ; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Further anticancer drugs that can be used in the methods and compositions of the invention include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin Im derivatives; balanol; batimastat; BCR/ABL antagonists; benzochlorins; benzoylstaurosporine; beta Lactam Derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox L-2; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomnifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-acytidine; dihydrotaxol; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum complexes; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agents; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum complexes; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In one another embodiment, the other anticancer agent is interferon-α.

In another embodiment, the other anticancer agent is interleukin-2.

In one embodiment, the other anticancer agent is an alkylating agent, such as a nitrogen mustard, a nitrosourea, an alkylsulfonate, a triazene, or a platinum-containing agent.

In another embodiment, the other anticancer agent is a triazene alkylating agent.

In a specific embodiment, the other anticancer agent is temozolomide.

Temozolomide can be administered to an animal at dosages ranging from about 60 mg/m² to about 250 mg/m² (of an animal's body surface area) and from about 100 mg/m² to about 200 mg/m². In specific embodiments, the dosages of temozolomide are about 10 mg/m, about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², or about 250 mg/m².

In a particular embodiment, temozolomide is administered orally.

In one embodiment, temozolomide is administered orally to an animal at a dose ranging from about 150 mg/m² to about 200 mg/m².

In another embodiment, temozolomide is administered orally to an animal once per day for five consecutive days at a dose ranging from about 150 mg/m² to about 200 mg/m².

In a specific embodiment, temozolomide is administered orally to an animal once per day for five consecutive days at a dose ranging from about 150 mg/m² to about 200 mg/m² on days 1-5, then again orally once per day for five consecutive days on days 28-32 at a dose ranging from about 150 mg/m² to about 200 mg/m², then again orally once per day for five consecutive days on days 55-59 at a dose ranging from about 150 mg/m² to about 200 mg/m².

In a specific embodiment, the other anticancer agent is procarbazine.

Procarbazine can be administered to a subject at dosages ranging from about 50 mg/m² (of a subject's body surface area) to about 100 mg/m² and from about 60 mg/m² to about 100 mg/m². In specific embodiments, the dosages of procarbazine are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m, about 260 mg/m², about 270 mg/m², about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m², about 450 mg/m², about 460 mg/m², about 470 mg/m², about 480 mg/m², about 490 mg/m², or about 500 mg/m².

In a particular embodiment, procarbazine is administered intravenously.

In one embodiment, procarbazine is administered intravenously to a subject at a dose ranging from about 50 mg/m² to about 100 mg/m².

In another embodiment, procarbazine is administered intravenously to a subject once per day for five consecutive days at a dose ranging from about 50 mg/m² to about 100 mg/m ².

In a specific embodiment, procarbazine is administered intravenously to a subject once per day for five consecutive days at a dose ranging from about 50 mg/m² to about 100 mg/m² on days 1-5, then again intravenously once per day for five consecutive days on days 28-32 at a dose ranging from about 50 mg/m² to about 100 mg/m², then again intravenously once per day for five consecutive days on days 55-59 at a dose ranging from about 50 mg/m² to about 100 mg/m².

In another embodiment, procarbazine is administered intravenously once to a subject at a dose ranging from about 50 mg/m² to about 100 mg/m².

In a specific embodiment, the other anticancer agent is dacarbazine.

Dacarbazine can be administered to a subject at dosages ranging from about 60 mg/m² (of a subject's body surface area) to about 250 mg/m² and from about 150 mg/m² to about 250 mg/m². In specific embodiments, the dosages of dacarbazine are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m², about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m² about 450 mg/m², about 460 mg/m², about 470 mg/m², about 480 mg/m², about 490 mg/m², or about 500 mg/m².

In a particular embodiment, dacarbazine is administered intravenously.

In one embodiment, dacarbazine is administered intravenously to a subject at a dose ranging from about 150 mg/m² to about 250 mg/m².

In another embodiment, dacarbazine is administered intravenously to a subject once per day for five consecutive days at a dose ranging from about 150 mg/m² to about 250 mg/m².

In a specific embodiment, dacarbazine is administered intravenously to a subject once per day for five consecutive days at a dose ranging from about 150 mg/m² to about 250 mg/m 2 on days 1-5, then again intravenously once per day for five consecutive days on days 28-32 at a dose ranging from about 150 mg/m² to about 250 mg/m², the n again intravenously once per day for five consecutive days on days 55-59 at a dose ranging from about 150 mg/m 2 to about 250 mg/m².

In one embodiment, dacarbazine is administered intravenously once to a subject at a dose ranging from about 150 mg/m² to about 250 mg/m².

In one embodiment, the other anticancer agent is a Topoisomerase I inhibitor, such as etoposide, teniposide, topotecan, irinotecan, 9-aminocamptothecin, camptothecin, or crisnatol.

In a specific embodiment, the other anticancer agent is irinotecan.

Irinotecan can be administered to a subject at dosages ranging from about 50 mg/m² (of a subject's body surface area) to about 150 mg/m² and from about 75 mg/m² to about 150 mg/m². In specific embodiments, the dosages of irinotecan are about 10 mg/m², about 1 mg/m 2, about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m, about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m², about 450 mg/m², about 460 mg/m², about 470 mg/m², about 480 mg/m², about 490 mg/m², or about 500

In a particular embodiment, irinotecan is administered intravenously.

In one embodiment, irinotecan is administered intravenously to a subject at a dose ranging from about 150 mg/m² to about 150 mg/m².

In another embodiment, irinotecan is administered intravenously to a subject once per day for five consecutive days at a dose ranging from about 150 mg/m² to about 150 mg/m².

In a specific embodiment, irinotecan is administered intravenously to a subject once per day for five consecutive days at a dose ranging from about 50 mg/m² to about 150 mg/m² on days 1-5, then again intravenously once per day for five consecutive days on days 28-32 at a dose ranging from about 50 mg/m 2 to about 150 mg/m², then again intravenously once per day for five consecutive days on days 55-59 at a dose ranging from about 50 mg/m² to about 150 mg/m².

In one embodiment, the invention provides administration of an effective a mount of: (i) a Tetracyclic Lactam Derivative and (ii) one or more other anticancer agents.

In one embodiment, (i) a Tetracyclic Lactam Derivative and (ii) one or more other anticancer agents are administered in doses commonly employed when such agents are used as monotherapy for the treatment of cancer.

In another embodiment, (i) a Tetracyclic Lactam Derivative and (ii) one or more other anticancer agents act synergistically and are administered in doses that are less than the doses commonly employed when such agents are used as monotherapy for the treatment of cancer.

The dosage of the (i) a Tetracyclic Lactam Derivative and (ii) one or more other anticancer agents administered as well as the dosing schedule can depend on various parameters, including, but not limited to, the cancer being treated, the patient's general health, and the administering physician's discretion.

In one embodiment, the other anticancer agent is O-6-benzylguanine.

In another embodiment, the other anticancer agent is O-6-benzylguanine and temozolomide.

In another embodiment, the other anticancer agent is O-6-benzylguanine and procarbazine.

In still another embodiment, the other anticancer agent is O-6-benzylguanine and dacarbazine.

### 4.10.11.1 Multi-Therapy for Cancer

The Tetracyclic Lactam Derivatives can be administered to an animal that has undergone or is currently undergoing one or more additional anticancer therapies including, but not limited to, surgery, radiation therapy, or immunotherapy, such as cancer vaccines.

In one embodiment, the invention provides methods for treating or preventing cancer, comprising administering to an animal in need thereof (a) an amount of a Tetracyclic Lactam Derivative effective to treat or prevent cancer; and (b) another anticancer therapy including, but not limited to, surgery, radiation therapy, or immunotherapy, such as a cancer vaccine.

In one embodiment, the other anticancer therapy is radiation therapy.

In another embodiment, the other anticancer therapy is surgery.

In still another embodiment, the other anticancer therapy is immunotherapy.

In a specific embodiment, the present methods for treating or preventing cancer comprise administering (i) a Tetracyclic Lactam Derivative and (ii) radiation therapy. The radiation therapy can be administered prior to, concurrently with, or subsequent to the Tetracyclic Lactam Derivative, in one embodiment, at least an hour, five hours, 12 hours, a day, a week, a month, in another embodiment, several months (e.g., up to three months), prior or subsequent to administration of the Tetracyclic Lactam Derivatives.

Where the other anticancer therapy is radiation therapy, any radiation therapy protocol can be used depending upon the type of cancer to be treated. For example, but not by way of limitation, X-ray radiation can be administered; in particular, high-energy megavoltage (radiation of greater that 1 MeV energy) can be used for deep tumors, and electron beam and orthovoltage X-ray radiation can be used for skin cancers. Gamma-ray emitting radioisotopes, such as radioactive isotopes of radium, cobalt and other elements, can also be administered.

Additionally, the invention provides methods of treatment of cancer using a Tetracyclic Lactam Derivatives as an alternative to chemotherapy or radiation therapy where the chemotherapy or the radiation therapy results in negative side effects, in the animal being treated. The animal being treated can, optionally, be treated with another anticancer therapy such as surgery, radiation therapy, or immunotherapy.

The Tetracyclic Lactam Derivative can also be used in vitro or ex vivo, such as for the treatment of certain cancers, including, but not limited to leukemias and lymphomas, such treatment involving autologous stem cell transplants. This can involve a process in which the animal's autologous hematopoietic stem cells are harvested and purged of all cancer cells, the animal's remaining bone-marrow cell population is then eradicated via the administration of a Tetracyclic Lactam Derivative and/or radiation, and the resultant stem cells are infused back into the animal. Supportive care can be subsequently provided while bone marrow function is restored and the animal recovers.

A Tetracyclic Lactam Derivative and the other therapeutic agent can act additively or, in one embodiment synergistically. In one embodiment a Tetracyclic Lactam Derivative is administered concurrently with another therapeutic agent. In one embodiment a composition comprising an effective amount of a Tetracyclic Lactam Derivative and an effective amount of another therapeutic agent can be administered. Alternatively, a composition comprising an effective amount of a Tetracyclic Lactam Derivative and a different composition comprising an effective amount of another therapeutic agent can be concurrently administered. In another embodiment an, effective amount of a Tetracyclic Lactam Derivative is administered prior or subsequent to administration of an effective amount of another therapeutic agent. In this embodiment the Tetracyclic Lactam Derivative is administered while the other therapeutic agent exerts its therapeutic effect, or the other therapeutic agent is administered while the Tetracyclic Lactam Derivative exerts its preventative or therapeutic effect for treating or preventing a Condition.

A composition of the invention is prepared by a method comprising admixing a Tetracyclic Lactam Derivative or a pharmaceutically acceptable salt and a physiologically acceptable carrier or vehicle. Admixing can be accomplished using methods well known for admixing a compound (or salt) and a physiologically acceptable carrier or vehicle. In one embodiment the Tetracyclic Lactam Derivative or the pharmaceutically acceptable salt of the Compound is present in the composition in an effective amount.

### 4.10.12 Kits

The invention encompasses kits that can simplify the administration of a Tetracyclic Lactam Derivative to an animal.

A typical kit of the invention comprises a unit dosage form of a Tetracyclic Lactam Derivative. In one embodiment the unit dosage form is a container, which can be sterile, containing an effective amount of a Tetracyclic Lactam Derivative and a physiologically acceptable carrier or vehicle. The kit can further comprise a label or printed instructions instructing the use of the Tetracyclic Lactam Derivative to treat or prevent a Condition. The kit can also further comprise a unit dosage form of another prophylactic or therapeutic agent, for example, a container containing an effective amount of the other prophylactic or therapeutic agent. In one embodiment the kit comprises a container containing an effective amount of a Tetracyclic Lactam Derivative and an effective amount of another prophylactic or therapeutic agent. Examples of other prophylactic or therapeutic agents include, but are not limited to, those listed above.

Kits of the invention can further comprise a device that is useful for administering the unit dosage forms. Examples of such a device includes, but is not limited to, a syringe, a drip bag, a patch, an inhaler, and an enema bag.

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### 5. EXAMPLES

### 5.1 Preparation of Illustrative Tetracyclic Lactam Derivatives

### 5.1.1 General Methods

Proton NMR spectra were obtained using a Varian 300 MHz spectrophotometer and chemical shift values (δ) are reported in parts per million (ppm). TLC was performed using TLC plates precoated with silica gel 60 F-254, and preparative TLC was performed using precoated Whatman 60A TLC plates. All intermediates and final compounds were characterized on the basis of ¹H NMR and/or MS data.

### 5.1.2 PREPARATION OF 4-PHENYL-3-ISOCOUMARINCARBOXYLIC ACID (102):

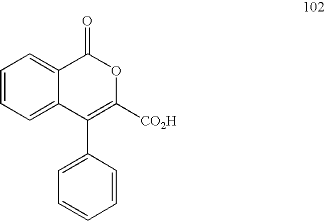

Following a literature procedure (Natsugary et al, J. Med. Chem. 1995, 38, 3106-3120), Compound 102 was synthesized. A suspension of 2-benzoyl-benzoic acid (33.9 g, 0.15 mol), anhydrous potassium carbonate (41.4 gm, 0.3 mol) and diethyl bromomalonate ((28.17 mL, 0.165 mol) in DMF (250 mL) was allowed to stir overnight at room temperature. The reaction mixture was then poured on cold water, and extracted with ethyl acetate. The organic layer was dried over sodium sulphate and concentrated. The residue obtained was treated with acetic acid (1.0 L), followed by concentrated HCl (800 mL), and then refluxed for 6 hours. The reaction mixture was cooled to room temperature and poured on ice cold water, and the precipitate that formed was filtered, washed thoroughly with water, and dried to provide 32.6 g of Compound 102, a white solid, in 84% yield.

### 5.1.3 PREPARATION OF 4-PHENYL-3-ISOQUINOLINONECARBOXYLIC ACID (103)

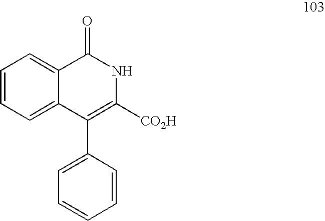

A stirred suspension of Compound 102 (1.4 g, 0.0052 mol) in ammonia-methanol (7N, 125 mL) was refluxed for 23 hours. The homogeneous reaction mixture was concentrated, and the residue obtained was acidified with diluted HCl. The solid precipitate was filtered, washed with water, and dried under vacuum to provide Compound 103 (1.225 g, 89%).

### 5.1.4 PREPARATION OF 3-OXOINDENO[2,1-C]ISOQUINOLINONE (104)

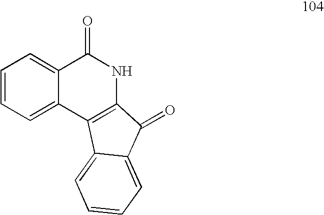

To a stirred suspension of Compound 103 (0.225 g, 0.85 mmol) in xylene (20 mL) was added polyphosphoric acid (0.600 gm). The reaction mixture was refluxed at 140-160° C. for 6 hours. Xylene was separated from the residue, and residue was poured onto ice. The resultant solid was filtered, washed with water, and dried to provide Compound 104 (155 mg, 74%).

Alternatively, Compound 103 (500 mg, 0.0019 mol) was reacted with chlorosulphonic acid (2.5 ml) at 0° C. for 5 minutes, and the reaction mixture was allowed to stir at room temperature for 5 minutes. After the reaction mixture became homogeneous, it was slowly poured onto ice. The red precipitate was filtered, washed with water, and dried to provide Compound 104 (395 mg, 85%).

### 5.1.5 PREPARATION OF 3-OXOINDENO[2,1-C]ISOQUINOLINONE HYDRAZONE (COMPOUND 1)

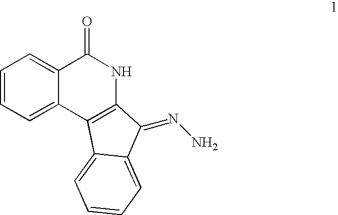

To a mixture of Compound 104 (110 mg) and hydrazine monohydrate (0.1 ml) in methanol (10 ml) was added concentrated HCl (0.1 ml) at room temperature. The reaction mixture was refluxed overnight. The precipitate was filtered, washed with water, and dried under vacuum to provide Compound 1 (35 mg). MS (ES⁺): m/z 262.2 (M+1).

### 5.1.6 PREPARATION OF [(3-OXOINDENO[2,1-C]ISOQUINOLINONE)-2-CYANOETHYL]-HYDRAZONE (COMPOUND 7)

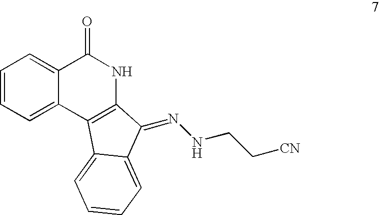

To a mixture of compound 104 (150 mg) and 2-cyanoethyl hydrazine (0.3 ml) was added acetic acid (10 ml) at room temperature. The reaction mixture was then refluxed overnight. The reaction mixture was concentrated in vacuo and the residue was treated with methanol (25 ml). The precipitate was filtered, washed with methanol and water, and dried under vacuum to provide Compound 7 (115 mg). ¹H-NMR (DMSO-d₆): 3.15 (t, J=6.6 Hz, 2H), 3.62-3.68 (m, 2H), 7.22 (t, J=7.5 Hz, 1H), 7.37 (t, J=7.5 Hz, 1H), 7.48 (t, J=8.1 Hz, 1H), 7.80 (t, J=7.5 Hz, 1H), 8.03 (d, J=7.8 Hz, 2H), 8.30 (d, J=7.8 Hz, 2H), 8.95 (s, 1H), 11.63 (s, 1H).

### 5.1.7 PREPARATION OF 3-OXOINDENO[2,1-C]ISOQUINOLINONE N-MORPHOLINO-HYDRAZONE (COMPOUND 105)

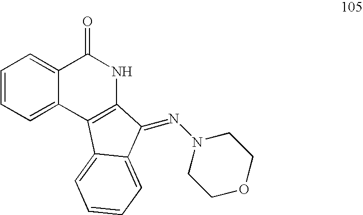

To a mixture of Compound 104 (75 mg) and N-morpholino hydrazine (0.3 ml) in ethanol (15 ml) was added concentrated hydrochloric acid (0.050 ml) at room temperature. The reaction mixture was refluxed for 67 hours. The reaction mixture was concentrated in vacuo and the residue was diluted with water and ethyl acetate (25 ml each), then neutralized with sodium bicarbonate. The organic layer was separated, concentrated and dried under vacuum to provide Compound 105 (48 mg).

### 5.1.8 PREPARATION OF 3-OXOINDENO[2,1-C]ISOOUINOLINONE N-(N-METHYLPIPERAZINO)-HYDRAZONE (COMPOUND 106)

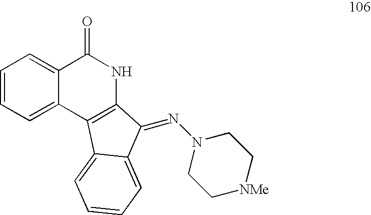

To a mixture of Compound 104 (75 mg) and N-(N-methyl-piperazine) hydrazine (5 eq) in ethanol (15 ml) was added concentrated hydrochloric acid (0.050 ml) at room temperature. The reaction mixture was then refluxed for 6-7 hours. The reaction mixture was concentrated in vacuo and the residue was diluted with water and ethyl acetate (25 ml each), then neutralized with sodium bicarbonate. The organic layer was separated, concentrated and dried under vacuum to provide Compound 106 (55 mg). ¹H-NMR (DMSO-d₆): 2.24 (s, 3H), 2.57-2.60)m, 4H), 3.25-3.28 (m, 4H), 7.27 (t, J=7.5 Hz, 1H), 7.40 (t, J=7.5 Hz, 1H), 7.55 (t, J=7.5 Hz, 1H), 7.68 (d, J=7.5 Hz, 1H), 7.82 (t, J=6.9 Hz, 1H), 7.99 (d, J=7.5 Hz, 1H), 8.30-8.34 (m, 2H), 11.48 (s, 1H).

### 5.1.9 PREPARATION OF ETHYL-5-OXO-5,6-DIRYDRO-INDOLO[3,2-C]ISOQUINOLINE-11-CARBOXYLATE (COMPOUND 63)

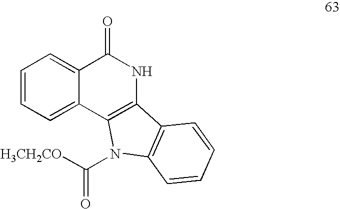

Homophthalic acid (50 g, 0.28 mol) was diluted with methanol (750 mL) and to the resultant solution was added sulfuric acid (3.75 mL, 5% v/v). The reaction mixture was heated at reflux for 24 hours under an inert atmosphere, then cooled to 5° C. To the resultant mixture was added dropwise 5N sodium hydroxide (28 mL) with vigorous stirring. The reaction mixture was concentrated in vacuo, and the resultant oil was diluted with ethyl acetate (200 mL) and sequentially washed using water (100 mL), saturated aqueous sodium carbonate (300 mL), water (300 mL) and brine (300 mL). The organic phase was dried over sodium sulfate, filtered, and concentrated in vacuo to provide dimethyl homophthalate as a light brown oil. Yield=39.4 g (68%).

Dimethyl homophthalate (19.27 g, 92.6 mmol) was diluted with benzene (300 mL), and to the resultant solution was added N-bromosuccinimide (21.43 g, 1.3 eq.). The reaction mixture was heated to reflux using a 500 Watt quartz halogen lamp. After nine hours at reflux, the reaction mixture was cooled to 6° C., then vacuum filtered through a glass frit. The filtrate was washed using saturated aqueous sodium carbonate (2×200 mL), then brine (200 mL). The organic phase was dried over sodium sulfate, filtered, and concentrated in vacuo to provide α-bromodiethyl homophthalate as a brown oil. Yield=26.59 g, (100%).

Anthranilonitrile (100.0 g, 0.85 mol) was diluted with pyridine (850 mL) and the resultant solution was cooled to 0° C. Ethyl chloroformate (85 mL, 1.05 eq.) was added dropwise over one hour and the reaction mixture was stirred at room temperature for 16 hours, then concentrated in vacuo to provide an off-white oily solid residue. To the off-white oily solid residue was added 0.5N aqueous HCl (1 L), and the resulting slurry was mechanically stirred for 1 hour, then filtered through #1 filter paper. The filtered solids were washed with water (2×IL), then dried in a vacuum oven for 96 hours. The dried solids were diluted with toluene (500 mL), and the resultant solution was distilled for 4 hours, during which time 300 mL of toluene was removed from the solution. The concentrated distillate was allowed to cool to room temperature and then was further cooled to 0° C. The resultant crystalline precipitate was filtered, then diluted with hexanes (250 mL). The resultant solution was allowed to stir at room temperature for 2 hours to provide a slurry, which was filtered through #1 filter paper. The collected solids were washed in the filter paper using hexanes (200 mL). The solids were then vacuum dried at room temperature to provide ethyl-N-(2-cyanophenyl)carbamate as a white crystalline solid. Yield=117.89 g (73%).

A 60% suspension of sodium hydride in oil (2.79 g, 2.0 eq.) was diluted with toluene (10 mL). To the resulting suspension was added a solution of ethyl-N-(2-cyanophenyl)carbamate in toluene (100 mL) via cannula. The cannula was washed using toluene (2×10 mL). To the resulting reaction mixture was added a solution of α-bromodimethyl homophthalate in toluene (40 mL) dropwise via cannula, and the resultant reaction mixture was stirred at reflux for 4 hours. The reaction mixture was then cooled to 0° C. and and 1N aqueous HCl (70 mL, 2.0 eq.) was added dropwise under an inert atmosphere. The resultant suspension was poured into a flask containing acetonitrile (200 mL) and stirred vigorously for 10 minutes. The resultant slurry was vacuum filtered, and the collected white solid was washed using acetonitrile (500 mL). The solid was dried in a vacuum oven at 400° C. to provide compound 63 as a white solid. Yield=5.0 g (47%).

### 5.1.10 PREPRARATION OF N-PROPYL-5-OXO-5,6-DIHYDRO-INDOLO[3,2-C]ISOQUINOLINE-11-CARBOXYLATE (COMPOUND 107)

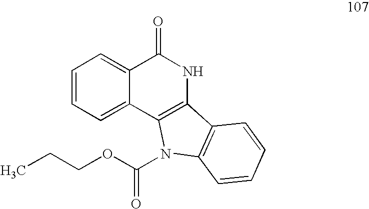

Following, the methodology of Radl, S., Konvicka, P., Vachal, P. J. Heterocycl. Chem. 2000, 37, 855-62 and Garcia, E. E.; Benjamin, L. E., Fryer, R. I. J. Heterocycl. Chem. 1973, 10, 51-3, solid n-propyl N-(2-cyanophenyl)carbamate (5.0 g, 24.5 mmol) was added to a stirring suspension of sodium hydride (60% dispersion in oil, 1.3 g, 32.8 mmol) in dry toluene (90 mL) at room temperature under nitrogen. After 5 minutes a solution of α-bromodimethylhomophthalate (4.7 g, 16.4 mmol) in dry toluene (10 mL) was added via syringe. The resultant mixture was heated to reflux for 6 hour. The reaction mixture was cooled to 10° C., and to it was added 1.0 N HCl (50 mL, 50 mmol) and acetonitrile (50 mL). The resultant suspension was filtered and the filtered solid was washed with acetonitrile (2×10 mL). The off-white solid was returned to the flask, washed by stirring in water (40 mL), and then collected via vacuum filtration. The dry solid was heated in refluxing acetonitrile (40 mL) for 8 hours, which was subsequently cooled to 10° C. The solid was collected via vacuum filtration to yield 3.8 g (51%) of Compound 107, an off-white powder: ¹H-NMR (300 MHz, d₆-DMSO) 12.46 (s, 1H), 8.38 (d, 1H), 8.19 (d, 1H), 8.11 (d, 2H), 7.78 (t, 1H), 7.58-7.44 (m, 2H), 7.40 (t, 1H), 4.49-4.41 (t, 2H), 1.86-1.75 (m, 2H), 0.99-0.88 (t, 3H); MS (ESI) m/z 321 (M+1).

### 5.1.11 PREPARATION OF ISO-PROPYL-5-OXO-5,6-DIHYDRO-INDOLO[3,2-C]ISOQUINOLINE-11-CARBOXYLATE (COMPOUND) 108)

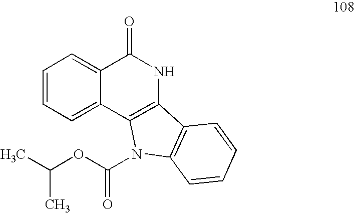

Following the above procedure for making Compound 107, 1.6 g (61%) of Compound 108, an off-white powder, was obtained, substituting isopropyl N-(cyanophenyl)carbamate and for n-propyl N-(2-cyanophenyl)carbamate: ¹H-NMR (300 MHz, d₆-DMSO) 12.49 (s, 1H), 8.32 (d, 1H), 8.16 (d, 1H), 8.12 (d, 2H), 7.78 (t, 1H), 7.58-7.48 (m, 2H), 7.38 (t, 1H), 5.33-5.21 (m, 1H), 1.42 (d, 6H); MS (ESI) m/z 321 (M+1).

### 5.1.12 PREPARATION OF N-BUTYL-5-OXO-5,6-DIHYDRO-INDOLO[3,2-C]ISOQUINOLINE-11-CARBOXYLATE (COMPOUND 109)

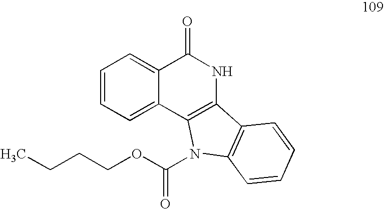

Following the above procedure for making Compound 107, 1.9 g (44%) of Compound 109, an off-white powder, was obtained, substituting n-butyl N-(cyanophenyl)carbamate for n-propyl N-(2-cyanophenyl)carbamate: ¹H-NMR (300 MHz, d₆DMSO) 12.52 (s, 1H), 8.37 (d, 1H), 8.19 (d, 1H), 8.12-8.07 (m, 2H), 7.81-7.75 (m, 1H), 7.58-7.50 (m, 2H), 7.41 (t, 1H), 4.50 (t, 2H), 1.81-1.69 (m, 2H), 1.44-1.35 (m, 2H), 0.91 (t, 3H); MS (ESI) m/z 335 (M+1).

### 5.1.13 PREPARATION OF TERT-BUTYL-5-OXO-5,6-DIHYDRO-INDOLO[3,2-C]ISOQUINOLINE-11-CARBOXYLATE (COMPOUND 62)

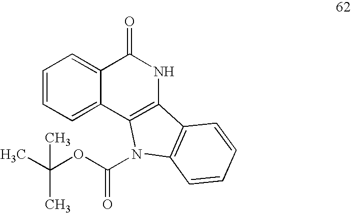

Following the above procedure for making Compound 107, 1.5 g (33%) of Compound 62, an off-white powder, was obtained, substituting tert-butyl N-(cyanophenyl)carbamate for n-propyl N-(2-cyanophenyl)carbamate: ¹H-NMR (300 MHz, d₆-DMSO) δ 12.48 (s, 1H), 8.58 (d, 1H), 8.19 (d, 1H), 8.11 (d, 1H), 7.99 (t, 1H), 7.78-7.64 (m, 2H), 7.47 (t, 1H), 7.29 (t, 1H), 1.57 (s, 9H); MS (ESI) m/z 335 (M+1).

### 5.1.14 PREPARATION OF ISO-BUTYL-5-OXO-5,6-DIHYDRO-INDOLO[3,2-C]ISOQUINOLINE-11-CARBOXYLATE (COMPOUND 110)

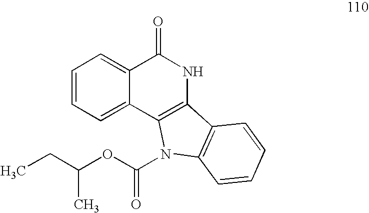

Following the above procedure for making Compound 107, 0.8 g (51%) of Compound 110, an off-white powder, was obtained, substituting isobutyl N-(cyanophenyl)carbamate for n-propyl N-(2-cyanophenyl)carbamate: ¹H-NMR (300 MHz, d₆-DMSO): 12.49 (s, 1H), 8.42 (d, 1H), 8.18 (d, 1H), 8.10 (d, 2H), 7.99 (t, 1H), 7.77-7.64 (m, 2H), 7.42 (t, 1H), 5.14-4.91 (d, 2H), 2.25-2.08 (m, H), 1.09-0.98 (m, 6H); MS (ESI) m/z 335 (M+1).

### 5.1.15 PREPARATION OF METHYL-5-OXO-5,6-DIHYDRO-INDOLO[3,2-C]ISOQUINOLINE-11-CARBOXYLATE (COMPOUND 61)

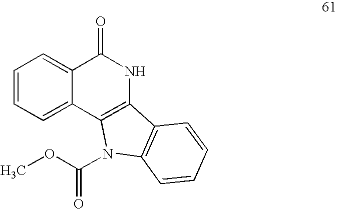

Following the above procedure for making Compound 107, 90 mg (18%) of Compound 61, an off-white powder, was obtained, substituting methyl N-(cyanophenyl)carbamate for n-propyl N-(2-cyanophenyl)carbamate: ¹H-NMR (300 MHz, d₆-DMSO) 12.44 (s, 1H), 8.38 (d, 1H), 8.18 (d, 1H), 8.12 (d, 2H), 7.78 (t, 1H), 7.59-7.48 (m, 2H), 7.38 (t, 1H), 4.09 (s, 3H); MS (ESI) m/z 293 (M+1).

### 5.1.16 PREPARATION OF N,N-DIMETHYL-5-OXO-5,6-DIHYDRO-INDOLO[3,2-C]ISOQUINOLINE-11-AMIDE (COMPOUND 94)

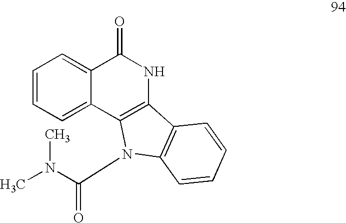

Following the above procedure for making Compound 107, 198 mg (9%) of Compound 94, an off-white powder, was obtained, substituting N′,N′-dimethyl N-(cyanophenyl)urea for n-propyl N-(2-cyanophenyl)carbamate: ¹H-NMR (300 MHz, d₆-DMSO) 12.44 (s, 1H), 8.39 (d, 1H), 8.16 (d, 1H), 7.81 (t, 1H), 7.78 (t, 1H), 7.69 (d, 1H), 7.56 (t, 1H), 7.42 (s, 2H), 7.37-7.24 (m, 1H), 3.24 (s, 3H), 3.01 (s, 3H); MS (ESI) m/z 306 (M+1).

### 5.2 Effect of Tetracyclic Lactam Derivatives on PARP Activity in Cultured Macrophages, Using a Whole-Cell Based Assay and a Purified Enzyme Assay

Demonstration of the ability of Tetracyclic Lactam Derivatives to inhibit PARP and prevent peroxynitrite induced cytotoxicity can be shown using methods described in Virag et al., Br. J. Pharmacol., 1999, 126(3):769-77; and Immunology 1998, 94(3):345-55. Without being bound by theory, Applicants believe that Tetracyclic Lactam Derivatives that inhibit PARP are useful for treating or preventing a Condition.

In a typical procedure, RAW mouse macrophages are cultured in DMEM medium with high glucose and supplemented with 10% fetal bovine serum. Cells are used at 80% confluence in 12-well plates. Cells are pretreated with various concentrations (100 nM-1 μM) of a Tetracyclic Lactam Derivative for 10 min. Peroxynitrite, a prototypical oxidant which induces DNA single strand breakage, is used to induce PARP activation. In a typical assay, peroxynitrite is diluted in phosphate buffered saline (PBS) (pH 11.0) and added to the cells in a bolus of 50 μL. Cells are then incubated for 20 minutes. Peroxynitrite is decomposed by incubation for 30 min at pH 7.0, and used as a control. After the 20 minute incubation period, the cells are spun, the medium is aspirated and the cells are resuspended in 0.5 mL assay buffer (56 mM HEPES pH 7:5, 28 mM KCl, 28 mM NaCl, 2 mM MgCl₂, 0.01% w/v digitonin and 0.125 μM NAD⁺ and 0.5 μCi/ml ³H-NAD⁺). Following incubation in assay buffer, (10 min at 37° C.), PARP activity can be measured as follows: 200 μL ice cold 50% w/v TCA is added and the samples are incubated for 4 h at 4° C. Samples are then spun (10 min @ 10,000 g) and the resulting pellets are washed twice with ice cold 5% w/v TCA and solubilized overnight in 250 μL 2% w/v SDS/0.1 N NaOH at 37° C. The contents of the tubes are added to 6.5 mL ScintiSafe Plus scintillation liquid (Fisher Scientific) and radioactivity is determined using a liquid scintillation counter (Wallac, Gaithersburg, Md.).

The potency of inhibition on purified PARP enzyme is determined for Tetracyclic Lactam Derivatives and compared with that of 3-aminobenzamide, a prototypical benchmark PARP inhibitor. The assay is performed in 96 well ELISA plates according to instructions provided with a commercially available PARP inhibition assay kit (Trevigen, Gaithersburg, Md.). In a typical procedure, wells are coated with 1 mg/mL of histone (50 μl/well) at 4° C. overnight. Plates are then washed four times with PBS and then blocked by adding 50 μL Strep-Diluent (supplied with the Trevigen kit). After incubation (1 h, room temperature), the plates are washed four times with PBS. Appropriate solutions of PARP inhibitors, including Tetracyclic Lactam Derivatives, are combined with 2×PARP cocktail (1.95 mM NAD⁺, 50 μM biotinylated NAD⁺ in 50 mM TRIS pH 8.0, 25 mM MgCl₂) and high specific activity PARP enzyme (both are supplied with the kit) in a volume of 50 μL. The reaction is allowed to proceed for 30 min at room temperature. After washing four times in PBS, incorporated biotin is detected by peroxidase-conjugated streptavidin (1:500 dilution) and TACS Sapphire substrate.

Examples of the inhibitory effects of illustrative Tetracyclic Lactam Derivatives in the whole-cell macrophage assay are illustrated in Tables 3 and 4 below.

TABLE 3
Inhibitory effect of illustrative Tetracyclic Lactam Derivatives
on PARP activation in cultured murine macrophages.

| | % PARP | % PARP | % PARP | % PARP |
|---|---|---|---|---|
| | Inhibition | Inhibition | Inhibition | Inhibition |
| Compound | at 3 μM | at 1 μM | at 0.3 μM | at 0.1 μM |
| 7 | NT | NT | NT | NT |
| 8 | NT | 84 | 75 | 60 |
| 63 | 65 | 50 | 43 | 33 |

TABLE 4
Inhibitory effect of illustrative Tetracyclic Lactam Derivatives
on PARP activation in cultured murine macrophages.

| Compound | IC₅₀ (μM) |
|---|---|
| 61 | 10 ≦ 100 |
| 63 | 1.0 ≦ 10 |
| 107 | 10 ≦ 100 |
| 108 | 10 ≦ 100 |
| 109 | 10 ≦ 100 |
| 110 | 10 ≦ 100 |

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

All references cited herein are incorporated by reference in their entirety.

1. A compound of the formula 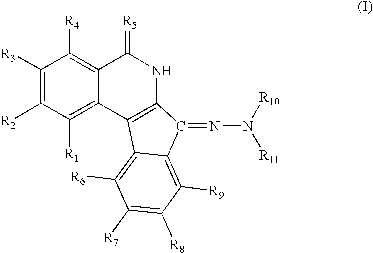
or a pharmaceutically acceptable salt thereof,
wherein:
R¹, R², R³, R⁴, R⁶, R⁷, R⁸ and R⁹ are independently —H, -halo, —OH, —NH₂, —CN, —NO₂, or -A-B;
R⁵ is O, S or NH;
A is —SO₂—, —SO₂NH—, —NHSO₂—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);
R¹⁰ is —H, —C₁-C₅ alkyl, —(CH₂)ₙ—CN, —(CH₂)ₙ-aryl, —(CH₂)ₙ-(3- to 7-membered monocyclic heterocycle), —(CH₂)ₙ-(7- to 10-membered bicyclic heterocycle), —(CH₂)ₙ—COO—(C₁-C₅ alkyl), —(CH₂)ₙ—COO-aryl, —(CH₂)ₙ—COOH, —CONH—(CH₂)ₙ—COOH, —CONH—(CH₂)ₙ—COO—(C₁-C₅ alkyl), —CONH—(CH₂)ₙ-aryl, —CONHNH—(C₁-C₅ alkyl), —CONHNH-aryl, —(CH₂)ₙ—CONH₂, —(CH₂)ₙ—CONH—(C₁-C₅ alkyl), —(CH₂)ₙ—CONH-aryl, —(CH₂)ₙ—CONH—(CH₂)_{q}-aryl, —(CH₂)ₙ—CONH—(CH₂)_{q}-(3- to 7-membered monocyclic heterocycle), —(CH₂)ₙ—CONH—(CH₂)_{q}-(7- to 10-membered monocyclic heterocycle), —(CH₂)ₙ—CONH—(CH₂)_{q}—CONH₂—(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)(CH₂)ₙ—(C₁-C₅ alkyl), —C(O)(CH₂)ₙ-aryl, —C(O)(CH₂)ₙ—COOH, —C(O)(CH₂)ₙ—COO—(C₁-C₅ alkyl), —C(O)(CH₂)ₙ—COO-(3- to 7-membered monocyclic heterocycle), —C(O)(CH₂)ₙ—COO-(7- to 10-membered bicyclic heterocycle), —C(O)(CH₂)ₙ-phenyl, —C(O)(CH₂)ₙ-(3- to 7-membered monocyclic heterocycle), —C(O)(CH₂)ₙ-(7- to 10-membered bicyclic heterocycle), —C(O)O(CH₂)ₙ-phenyl, —C(O)O(CH₂)ₙ-(3- to 7-membered monocyclic heterocycle), —C(O)O(CH₂)ₙ-(7- to 10-membered bicyclic heterocycle), —C(O)N((CH₂)ₙ-phenyl)₂, —C(O)N((CH₂)ₙ-phenyl)((CH₂)_{q}-3- to 7-membered monocyclic heterocycle), —C(O)N((CH₂)ₙ-phenyl)((CH₂)_{q} 7- to 10-membered bicyclic heterocycle), —C(O)N((CH₂)ₙ-(3- to 7-membered monocyclic heterocycle)₂, —C(O)N((CH₂)ₙ-7- to 10-membered bicyclic heterocycle)₂, or —SO₂NH₂;
R¹¹ is —H, or (—C₁-C₆ alkyl), or R¹⁰, R¹¹ and the nitrogen atom to which they are attached join to form a -(nitrogen-containing 3- to 7-membered monocyclic heterocycle), or a -(nitrogen-containing 7- to 10-membered bicyclic heterocycle);
each n is independently an integer ranging from 0 to 10;
each p is independently an integer ranging from 0 to 5; and
each q is independently an integer ranging from 0 to 10.

2. A compound of the formula 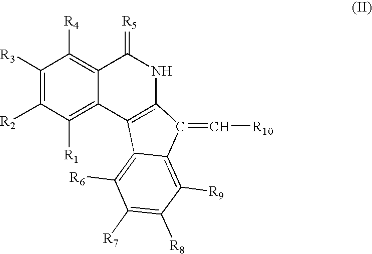
or a pharmaceutically acceptable salt thereof, wherein:
R¹, R², R³, R⁴, R⁶, R⁷, R⁸ and R⁹ are independently —H, -halo, —OH, —NH₂, —CN, —NO₂, or -A-B;
R⁵ is O, S or NH;
A is —SO₂—, —SO₂NH—, —NHSO₂—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(A), where Z₃ and Z are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z; and Z are taken together to form a -(nitrogen-containing-3- to 7-membered monocyclic heterocycle) or a -(nitrogen-containing 7- to 10-membered bicyclic heterocycle);
R¹⁰ is —H, —C₁-C₅ alkyl, —(CH₂)ₙ—CN, —(CH₂)ₙ-aryl, —(CH₂)ₙ-(3- to 7-membered monocyclic heterocycle), —(CH₂), -(7- to 10-membered bicyclic heterocycle), —(CH₂)ₙ—COO—(C₁-C₅ alkyl), —(CH₂)ₙ—COO-aryl, —(CH₂)ₙ—COOH, —CONH—(CH₂)ₙ—COOH, —CONH—(CH₂)ₙ—COO—(C₁-C₅ alkyl), —CONH—(CH₂)ₙ-aryl, —CONHNH—(C₁-C₅ alkyl), —CONHNHaryl, —(CH₂)ₙ—CONH₂, —(CH₂), —CONH—(C₁-C₅ alkyl), —(CH₂)ₙ—CONH-aryl, —(CH₂)ₙ—CONH—(CH₂)_{q}-aryl, —(CH₂)ₙ—CONH—(CH₂)_{q}-(3 to 7-membered monocyclic heterocycle), —(CH₂)ₙ—CONH—(CH₂)_{q}-(7- to 10-membered bicyclic heterocycle), —(CH₂)ₙ—CONH—(CH₂)_{q}—CONH₂—(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)(CH₂)ₙ—(C₁-C₅ alkyl), —C(O)(CH₂)ₙ-aryl, —C(O)(CH₂)ₙ—COOH, —C(O)(CH₂)ₙ—COO—(C₁-C₅ alkyl), —C(O)(CH₂)ₙ—COO-(3- to 7-membered monocyclic heterocycle), —C(O)(CH₂)ₙ—COO-(7- to 10-membered bicyclic heterocycle), —C(O)(CH₂)ₙ-phenyl, —C(O)(CH₂)ₙ-(3- to 7-membered monocyclic heterocycle), —C(O)(CH₂)ₙ-(7- to 10-membered bicyclic heterocycle), —C(O)O(CH₂)ₙ-phenyl, —C(O)O(CH₂)ₙ-(3- to 7-membered monocyclic heterocycle), —C(O)O(CH₂)ₙ-(7- to 10-membered bicyclic heterocycle), —C(O)N((CH₂)ₙ-phenyl)₂, —C(O)N((CH₂)ₙ-phenyl)((CH₂)_{q}-3- to 7-membered monocyclic heterocycle), —C(O)N((CH₂)ₙ-phenyl)((CH₂)_{q}-7- to 10-membered bicyclic heterocycle), —C(O)N((CH₂)ₙ-3- to 7-membered monocyclic heterocycle)₂, —C(O)N((CH₂)ₙ-7- to 10-membered bicyclic heterocycle)₂, or —SO₂NH₂;
each n is independently an integer ranging from 0 to 10;
each p is independently an integer ranging from 0 to 5; and
each q is independently an integer ranging from 0 to 10.

3. A compound of the formula 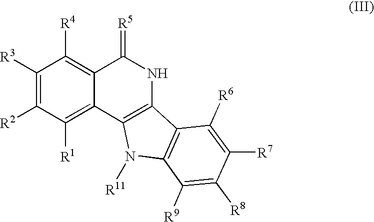
or a pharmaceutically acceptable salt thereof,
wherein:
R¹, R², R³, R⁴, R⁶, R⁷, R⁸ and R⁹ are each independently —H, —O—(C₁-C₅ alkyl), —C₁-C₁₀ alkyl, —C₂-C₁₀ alkenyl, -aryl, —C(O)OH, —C(O)O(C₁-C₅ alkyl), —OC(O)(C₁-C₅ alkyl), —NO₂, —NHC(O)(CH₂)ₙ—NH₂, —NHSO₂NH(CH₂)ₙ—NH₂, —C(O)NH(CH₂)ₙ—NH₂, —SO₂NH(CH₂)ₙ—NH₂, -halo, —OH, —NH₂, or -A-B;
R⁵ is O, S or NH;
A is —SO₂—, —SO₂NH—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);
R¹¹ is —H, —C₁-C₅ alkyl, —(CH₂)ₙ-aryl, —C(O)R¹², —C(O)OR¹², —C(O)O—(C₁-C₅ alkyl), —CONH₂, —C(O)NH—(CH₂)ₙ—C(O)OH, —(CH₂)ₙ—C(O)OH, —(CH₂)ₙ—CONH—(CH₂)_{q}-(3- to 7-membered monocyclic heterocycle), —(CH₂)ₚ-(3- to 7-membered bicyclic heterocycle), —(CH₂)ₚ-(7- to 10-membered bicyclic heterocycle), —(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)—(CH₂)ₙ—C(O)O—(C₁-C₅ alkyl), —CONH—(CH₂)ₚ-(3- to 7-membered monocyclic heterocycle), —C(O)N(R¹²)₂, —C(O)NHNHR¹², CONH(CH₂)ₙN(R¹²)₂, —CONHN(Z)(Z₂), or -A-B;
each occurrence of R¹² is independently —H, —(C₁-C₅ alkyl), —(CH₂)ₚ-phenyl, —(CH₂)ₚ-(3- to 7-membered monocyclic heterocycle), or —(CH₂)ₚ-7- to 10-membered bicyclic heterocycle;
each n is independently an integer ranging from 1 to 10;
each p is independently an integer ranging from 0 to 5; and
each q is independently an integer ranging from 0 to 10.

4. The compound of claim 1, wherein R¹, R², R³ and R⁴ are independently —H, —F, —OH, or —O—(C₁-C₅ alkyl).

5. The compound or pharmaceutically acceptable salt of the compound of claim 4, wherein R¹, R², R³ and R⁴ are each —H.

6. The compound or pharmaceutically acceptable salt of the compound of claim 1, wherein R¹, R², R³, R⁴, R⁶, R⁷, R⁸ and R⁹ are each hydrogen.

7. The compound or pharmaceutically acceptable salt of the compound of claim 1, wherein R⁵ is oxygen.

8. The compound or pharmaceutically acceptable salt of the compound of claim 7, wherein R¹, R², R³ and R⁴ are each hydrogen.

9. The compound or pharmaceutically acceptable salt of the compound of claim 7, wherein R⁶, R⁷, R⁸ or R⁹ is -A-B, where A is —NHC(O)— and B is —(C₁-C₅ alkylene)-NZ₁Z₂.

10. The compound or pharmaceutically acceptable salt of the compound of claim 7, wherein R⁶, R⁷, R⁸ or R⁹ is -A-B, where A is —SO₂NH—; B is —(C₁-C₁₀ alkyl)-N(Z₁)(Z₂); and N, Z₁ and Z₂ are taken together to form a nitrogen-containing 3- to 7-membered monocyclic heterocycle.

11. The compound or pharmaceutically acceptable salt of the compound of claim 9, wherein R⁸ is —NHC(O)CH₂N(CH₃)₂.

12. The compound or pharmaceutically acceptable salt of the compound of claim 10, wherein R⁸ is —SO₂NH(CH₂)₃-(morpholin-4-yl).

13. The compound or pharmaceutically acceptable salt of the compound of claim 7, wherein R¹⁰ is —H, -alkyl, —(CH₂)ₙ-aryl, —COO—(C₁-C₅ alkyl), —CONH₂, —CONH—(CH₂)ₙ—COOH, —(CH₂)ₙ—CONH(CH₂)_{q}-(3- to 7-membered monocyclic heterocycle), —(CH₂)ₙ—CONH—(CH₂)_{q}-(7- to 10-membered bicyclic heterocycle), —(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)—(C₁-C₅ alkyl) or —C(O)(CH₂)ₙ—COO—(C₁-C₅ alkyl).

14. The compound or pharmaceutically acceptable salt of the compound of claim 1, wherein R¹⁰, R¹¹, and the nitrogen to which they are attached form a -(nitrogen-containing 3- to 7-membered monocyclic heterocycle).

15. The compound or pharmaceutically acceptable salt of the compound of claim 2, wherein R¹, R², R³ and R⁴ are independently —H, —F, —OH, or —O—(C₁-C₅ alkyl).

16. The compound or pharmaceutically acceptable salt of the compound of claim 14, wherein R¹, R², R³ and R⁴ are each —H.

17. The compound or pharmaceutically acceptable salt of the compound of claim 2, wherein R¹, R², R³, R⁴, R⁶, R⁷, R⁸ and R⁹ are each hydrogen.

18. The compound or pharmaceutically acceptable salt of the compound of claim 2, wherein R⁵ is oxygen.

19. The compound or pharmaceutically acceptable salt of the compound of claim 18, wherein R¹, R², R³ and R⁴ are each hydrogen.

20. The compound or pharmaceutically acceptable salt of the compound of claim 18, wherein R⁶, R⁷, R⁸ or R⁹ is -A-B, where A is —NHC(O)— and B is —(C₁-C₅ alkylene)-NZ₁Z₂.

21. The compound or pharmaceutically acceptable salt of the compound of claim 18, wherein R⁶, R⁷, R⁸ or R⁹ is -A-B, where A is —SO₂NH—; B is —(C₁-C₁₀ alkyl)-N(Z₁)(Z₂); and N, Z₁ and Z₂ are taken together to form a nitrogen-containing 3- to 7-membered monocyclic heterocycle.

22. The compound or pharmaceutically acceptable salt of the compound of claim 20, wherein R⁸ is —NHC(O)CH₂N(CH₃)₂.

23. The compound or pharmaceutically acceptable salt of the compound of claim 21, wherein R⁸ is —SO₂NH(CH₂)₃-(morpholin-4-yl).

24. The compound or pharmaceutically acceptable salt of the compound of claim 18, wherein R¹⁰ is —H, —C₁-C₅ alkyl, —(CH₂)ₙ-aryl, —COO—(C₁-C₅ alkyl), —CONH₂, —CONH—(CH₂)ₙ—COOH, —(CH₂)ₙ—COOH, —(CH₂)ₙ—CONH—(CH₂)_{q}-(3 to 7-membered monocyclic heterocycle), —(CH₂)ₙ—CONH—(CH₂)_{q}-(7- to 10-membered bicyclic heterocycle), —(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)—(C₁-C₅ alkyl) or —C(O)(CH₂)ₙ—COO—(C₁-C₅ alkyl).

25. The compound or pharmaceutically acceptable salt of the compound of claim 3, wherein R¹, R², R³ and R⁴ are independently —H, —F, —OH, or —O—(C₁-C₅ alkyl).

26. The compound or pharmaceutically acceptable salt of the compound of claim 25, wherein R¹, R², R³ and R⁴ are each —H.

27. The compound or pharmaceutically acceptable salt of the compound of claim 3, wherein R¹, R², R³, R⁴, R⁶, R⁷, R⁸ and R⁹ are each hydrogen.

28. The compound or pharmaceutically acceptable salt of the compound of claim 3, wherein R⁵ is oxygen.

29. The compound or pharmaceutically acceptable salt of the compound of claim 28, wherein R¹, R², R³ and R⁴ are each hydrogen.

30. The compound or pharmaceutically acceptable salt of the compound of claim 28, wherein R⁷ is —H and R⁸ is -A-B, where A is —NHC(O)— and B is —(C₁-C₅ alkylene)-NZ₁Z₂.

31. The compound or pharmaceutically acceptable salt of the compound of claim 28, wherein R⁸ is —H and R⁷ is -A-B, where A is —NHC(O)— and B is —(C₁-C₅ alkylene)-NZ₁Z₂.

32. The compound or pharmaceutically acceptable salt of the compound of claim 28, wherein R⁷ is —H and R⁸ is -A-B, where A is —SO₂NH—; B is —(C₁-C₁₀ alkyl)-N(Z₁)(Z₂); and N, Z₁ and Z₂ are taken together to form a nitrogen-containing 3- to 7-membered monocyclic heterocycle.

33. The compound or pharmaceutically acceptable salt of the compound of claim 28, wherein R⁸ is —H and R⁷ is -A-B, where A is —SO₂NH—; B is —(C₁-C₁₀ alkyl)-N(Z₁)(Z₂); and N, Z₁ and Z₂ are taken together to form a nitrogen-containing 3- to 7-membered monocyclic heterocycle.

34. The compound or pharmaceutically acceptable salt of the compound of claim 30, wherein R⁷ is —H and R⁸ is —NHC(O)CH₂N(CH₃)₂.

35. The compound or pharmaceutically acceptable salt of the compound of claim 30, wherein R⁸ is —H and R⁷ is —NHC(O)CH₂N(CH₃)₂.

36. The compound or pharmaceutically acceptable salt of the compound of claim 31, wherein R⁷ is —H and R⁸ is —SO₂NH(CH₂)₃-(morpholin-4-yl).

37. The compound or pharmaceutically acceptable salt of the compound of claim 31, wherein R⁸ is —H and R⁷ is —SO₂NH(CH₂)₃-(morpholin-4-yl).

38. The compound or pharmaceutically acceptable salt of the compound of claim 28, wherein R¹¹ is —C(O)R¹², —C(O)OR¹², —C(O)NH—(CH₂)ₚ-(3- to 7-membered monocyclic heterocycle), —C(O)N(R¹²)₂, —C(O)NH(CH₂)ₙN(R¹²)₂, —C(O)NHNHR¹², —C(O)NH—N(Z₁)(Z₂), —(C₁-C₅ alkyl), —(CH₂)ₚ-phenyl, —(CH₂)ₚ-(3- to 7-membered monocyclic heterocycle), —(CH₂)ₚ-(7- to 10-membered bicyclic heterocycle), or -A-B.

39. The compound or pharmaceutically acceptable salt of the compound of claim 34, wherein R¹¹ is —COO—(C₁-C₅ alkyl), or —C(O)O—(C₁-C₅ alkyl)-NZ₁Z₂.

40. A composition comprising an effective amount of a compound or pharmaceutically acceptable salt of a compound of claim 1 and a physiologically acceptable carrier or vehicle.

41. A composition comprising an effective amount of a compound or pharmaceutically acceptable salt of a compound of claim 2 and a physiologically acceptable carrier or vehicle.

42. A composition comprising an effective amount of a compound or pharmaceutically acceptable salt of a compound of claim 3 and a physiologically acceptable carrier or vehicle.

43. A composition comprising an effective amount of a compound or pharmaceutically acceptable salt of a compound of claim 1, an effective amount of temozolomide, and a physiologically acceptable carrier or vehicle.

44. A composition comprising an effective amount of a compound or pharmaceutically acceptable salt of a compound of claim 2, an effective amount of temozolomide, and a physiologically acceptable carrier or vehicle.

45. A composition comprising an effective amount of a compound or pharmaceutically acceptable salt of a compound of claim 3, an effective amount of temozolomide, and a physiologically acceptable carrier or vehicle.

46. A composition comprising an effective amount of a compound or pharmaceutically acceptable salt of a compound of claim 1, an effective amount of procarbazine, and a physiologically acceptable carrier or vehicle.

47. A composition comprising an effective amount of a compound or pharmaceutically acceptable salt of a compound of claim 2, an effective amount of procarbazine, and a physiologically acceptable carrier or vehicle.

48. A composition comprising an effective amount of a compound or pharmaceutically acceptable salt of a compound of claim 3, an effective amount of procarbazine, and a physiologically acceptable carrier or vehicle.

49. A composition comprising an effective amount of a compound or pharmaceutically acceptable salt of a compound of claim 1, an effective amount of dacarbazine, and a physiologically acceptable carrier or vehicle.

50. A composition comprising an effective amount of a compound or pharmaceutically acceptable salt of a compound of claim 2, an effective amount of dacarbazine, and a physiologically acceptable carrier or vehicle.

51. A composition comprising an effective amount of a compound or pharmaceutically acceptable salt of a compound of claim 3, an effective amount of dacarbazine, and a physiologically acceptable carrier or vehicle.

52. A composition comprising an effective amount of a compound or pharmaceutically acceptable salt of a compound of claim 1, an effective amount of irinotecan, and a physiologically acceptable carrier or vehicle.

53. A composition comprising an effective amount of a compound or pharmaceutically acceptable salt of a compound of claim 2, an effective amount of irinotecan, and a physiologically acceptable carrier or vehicle.

54. A composition comprising an effective amount of a compound or pharmaceutically acceptable salt of a compound of claim 3, an effective amount of irinotecan, and a physiologically acceptable carrier or vehicle.

55. A composition comprising an effective amount of a compound or pharmaceutically acceptable salt of a compound of claim 1, an effective amount of Interleukin-2, and a physiologically acceptable carrier or vehicle.

56. A composition comprising an effective amount of a compound or pharmaceutically acceptable salt of a compound of claim 2, an effective amount of Interleukin-2, and a physiologically acceptable carrier or vehicle.

57. A composition comprising an effective amount of a compound or pharmaceutically acceptable salt of a compound of claim 3, an effective amount of Interleukin-2, and a physiologically acceptable carrier or vehicle.

58. A method for treating an inflammatory disease, comprising administering to an animal in need thereof an amount of a compound or a pharmaceutically acceptable salt of a compound of claim 1 effective to treat the inflammatory disease.

59. A method for treating an inflammatory disease, comprising administering to an animal in need thereof an amount of a compound or a pharmaceutically acceptable salt of a compound of claim 2 effective to treat the inflammatory disease.

60. A method for treating an inflammatory disease, comprising administering to an animal in need thereof an amount of a compound or a pharmaceutically acceptable salt of a compound of claim 3 effective to treat the inflammatory disease.

61. The method of claim 58, wherein the inflammatory disease is an inflammatory disease of a joint, a chronic inflammatory disease of the gum, an inflammatory bowel disease, an inflammatory lung disease, an inflammatory disease of the central nervous system, an inflammatory disease of the eye, gram-positive shock, gram negative shock, hemorrhagic shock, anaphylactic shock, traumatic shock or chemotherapeutic shock.

62. The method of claim 59, wherein the inflammatory disease is an inflammatory disease of a joint, a chronic inflammatory disease of the gum, an inflammatory bowel disease, an inflammatory lung disease, an inflammatory disease of the central nervous system, an inflammatory disease of the eye, gram-positive shock, gram negative shock, hemorrhagic shock, anaphylactic shock, traumatic shock or chemotherapeutic shock.

63. The method of claim 60, wherein the inflammatory disease is an inflammatory disease of a joint, a chronic inflammatory disease of the gum, an inflammatory bowel disease, an inflammatory lung disease, an inflammatory disease of the central nervous system, an inflammatory disease of the eye, gram-positive shock, gram negative shock, hemorrhagic shock, anaphylactic shock, traumatic shock or chemotherapeutic shock.

64. A method for treating a reperfusion injury, comprising administering to an animal in need thereof an amount of a compound or pharmaceutically acceptable salt of a compound of claim 1 effective to treat the reperfusion injury.

65. A method for treating a reperfusion injury, comprising administering to an animal in need thereof an amount of a compound or pharmaceutically acceptable salt of a compound of claim 2 effective to treat the reperfusion injury.

66. A method for treating a reperfusion injury, comprising administering to an animal in need thereof an amount of a compound or pharmaceutically acceptable salt of a compound of claim 3 effective to treat the reperfusion injury.

67. The method of claim 64, wherein the reperfusion injury is stroke or myocardial infarction.

68. The method of claim 65, wherein the reperfusion injury is stroke or myocardial infarction.

69. The method of claim 66, wherein the reperfusion injury is stroke or myocardial infarction.

70. A method for treating diabetes or a diabetic complication, comprising administering to an animal in need thereof an amount of a compound or pharmaceutically acceptable salt of a compound of claim 1 effective to treat diabetes or the diabetic complication.

71. A method for treating diabetes or a diabetic complication, comprising administering to an animal in need thereof an amount of a compound or pharmaceutically acceptable hydrate or salt of a compound of claim 2 effective to treat diabetes or the diabetic complication.

72. A method for treating diabetes or a diabetic complication, comprising administering to an animal in need thereof an amount of a compound or pharmaceutically acceptable salt of a compound of claim 3 effective to treat diabetes or the diabetic complication.

73. The method of claim 70, wherein the diabetes is Type I diabetes or Type II diabetes.

74. The method of claim 71, wherein the diabetes is Type I diabetes or Type II diabetes.

75. The method of claim 72, wherein the diabetes is Type I diabetes or Type II diabetes.

76. A method for treating cancer, comprising administering to an animal in need thereof an amount of a compound or pharmaceutically acceptable salt of a compound of claim 1 effective to treat cancer.

77. A method for treating cancer, comprising administering to an animal in need thereof an amount of a compound or pharmaceutically acceptable salt of a compound of claim 2 effective to treat cancer.

78. A method for treating cancer, comprising administering to an animal in need thereof an amount of a compound or pharmaceutically acceptable salt of a compound of claim 3 effective to treat cancer.

79. The method of claim 76, wherein the cancer is colorectal cancer, lung cancer, pancreatic cancer, esophageal cancer, stomach cancer, skin cancer, leukemia, lymphoma, non-Hodgkin's lymphoma, testicular cancer, bladder cancer, kidney cancer, liver cancer, breast cancer, prostate cancer, head and neck cancer, brain cancer, cancer of the central nervous system, uterine cancer, cervical cancer, or ovarian cancer.

80. The method of claim 77, wherein the cancer is colorectal cancer, lung cancer, pancreatic cancer, esophageal cancer, stomach cancer, skin cancer, leukemia, lymphoma, non-Hodgkin's lymphoma, testicular cancer, bladder cancer, kidney cancer, liver cancer, breast cancer, prostate cancer, head and neck cancer, brain cancer, cancer of the central nervous system, uterine cancer, cervical cancer, or ovarian cancer.

81. The method of claim 78, wherein the cancer is colorectal cancer, lung cancer, pancreatic cancer, esophageal cancer, stomach cancer, skin cancer, leukemia, lymphoma, non-Hodgkin's lymphoma, testicular cancer, bladder cancer, kidney cancer, liver cancer, breast cancer, prostate cancer, head and neck cancer, brain cancer, cancer of the central nervous system, uterine cancer, cervical cancer, or ovarian cancer.

82. The method of claim 76, wherein the cancer is or metastatic brain cancer, glioma, or melanoma.

83. The method of claim 77, wherein the cancer is metastatic brain cancer, glioma, or melanoma.

84. The method of claim 78, wherein the cancer is metastatic brain cancer, glioma, or melanoma.

85. The method of claim 82, wherein the glioma is piocytic astrocytma, astrocystoma, anaplastic astrocytoma, glioblastoma multiforme.

86. The method of claim 83, wherein the glioma is piocytic astrocytma, astrocystoma, anaplastic astrocytoma, glioblastoma multiforme.

87. The method of claim 84, wherein the glioma is piocytic astrocytma, astrocystoma, anaplastic astrocytoma, glioblastoma multiforme.

88. The method of claim 76, further comprising administering an effective amount of temozolomide, procarbazine, dacarbazine, irinotecan, Interleukin-2, or a combination thereof.

89. The method of claim 77, further comprising administering an effective amount of temozolomide, procarbazine, dacarbazine, irinotecan, Interleukin-2, or a combination thereof.

90. The method of claim 78, further comprising administering an effective amount of temozolomide, procarbazine, dacarbazine, irinotecan, Interleukin-2, or a combination thereof.

91. A method for treating renal failure, comprising administering to an animal in need thereof an amount of a compound or pharmaceutically acceptable salt of a compound of claim 1 effective to treat renal failure.

92. A method for treating renal failure, comprising administering to an animal in need thereof an amount of a compound or pharmaceutically acceptable salt of a compound of claim 2 effective to treat renal failure.

93. A method for treating renal failure, comprising administering to an animal in need thereof an amount of a compound or pharmaceutically acceptable salt of a compound of claim 3 effective to treat renal failure.

94. The method of claim 91, wherein the renal failure is chronic renal failure or acute renal failure.

95. The method of claim 92, wherein the renal failure is chronic renal failure or acute renal failure.

96. The method of claim 93, wherein the renal failure is chronic renal failure or acute renal failure.

97. A method for treating a vascular disease, comprising administering to an animal in need thereof an amount of a compound or pharmaceutically acceptable salt of a compound of claim 1 effective to treat the vascular disease.

98. A method for treating a vascular disease, comprising administering to an animal in need thereof an amount of a compound or pharmaceutically acceptable salt of a compound of claim 2 effective to treat the vascular disease.

99. A method for treating a vascular disease, comprising administering to an animal in need thereof an amount of a compound or pharmaceutically acceptable salt of a compound of claim 3 effective to treat the vascular disease.

100. The method of claim 97, wherein the vascular disease is a cardiovascular disease.

101. The method of claim 98, wherein the vascular disease is a cardiovascular disease.

102. The method of claim 99, wherein the vascular disease is a cardiovascular disease.

103. The method of claim 100, wherein the cardiovascular disease is chronic heart failure or a cardiac arrhythmia.

104. The method of claim 101, wherein the cardiovascular disease is chronic heart failure or a cardiac arrhythmia.

105. The method of claim 102, wherein the cardiovascular disease is chronic heart failure or a cardiac arrhythmia.

106. A method for treating an ischemic condition, comprising administering to an animal in need thereof an amount of a compound or pharmaceutically acceptable salt of a compound of claim 1 effective to treat the ischemic condition.

107. A method for treating an ischemic condition, comprising administering to an animal in need thereof an amount of a compound or pharmaceutically acceptable salt of a compound of claim 2 effective to treat the ischemic condition.

108. A method for treating an ischemic condition, comprising administering to an animal in need thereof an amount of a compound or pharmaceutically acceptable salt of a compound of claim 3 effective to treat the ischemic condition.

109. The method of claim 106, wherein the ischemic condition is myocardial ischemia, stable angina, unstable angina, stroke, ischemic heart disease or cerebral ischemia.

110. The method of claim 107, wherein the ischemic condition is myocardial ischemia, stable angina, unstable angina, stroke, ischemic heart disease or cerebral ischemia.

111. The method of claim 108, wherein the ischemic condition is myocardial ischemia, stable angina, unstable angina, stroke, ischemic heart disease or cerebral ischemia.

112. A method of treating a reoxygenation injury resulting from organ transplantation, comprising administering to an animal in need thereof an effective amount of a compound or pharmaceutically acceptable salt of the compound of claim 1.

113. A method of treating a reoxygenation injury resulting from organ transplantation, comprising administering to an animal in need thereof an effective amount of a compound or pharmaceutically acceptable salt of the compound of claim 2.

114. A method of treating a reoxygenation injury resulting from organ transplantation, comprising administering to an animal in need thereof an effective amount of a compound or pharmaceutically acceptable salt of the compound of claim 3.

115. A method of treating Parkinson's disease, comprising admininstering to an animal in need thereof an effective amount of a compound or pharmaceutically acceptable salt of the compound of claim 1.

116. A method of treating Parkinson's disease, comprising admininstering to an animal in need thereof an effective amount of a compound or pharmaceutically acceptable salt of the compound of claim 2.

117. A method of treating Parkinson's disease, comprising admininstering to an animal in need thereof an effective amount of a compound or pharmaceutically acceptable salt of the compound of claim 3.
